(19) United States
(12) Patent Application Publication
(10) Pub. No.: US 2003/0099725 A1
(43) Pub. Date: May 29, 2003

(54) Herbal compositions useful as chemopreventive and therapeutic agents and methods of manufacturing same

(75) Inventors: John Wah Tze, Vancouver (CA); Peizhong Lin, Beijing (CN); Stephen Lam, Vancouver (CA); Joseph Tai, Vancouver (CA)

Correspondence Address: Michael L. Goldman, NIXON PEABODY LLP, Clinton Square, P.O. Box 31051, Rochester, NY, 14603, US

(73) Assignee: Global Cancer Strategies Ltd.

(21) Appl. No.: 09/793,251

(22) Filed: Feb. 26, 2001

(51) Int. Cl. A61K 35/78

(52) U.S. Cl. 424/725; 424/765; 424/764; 424/728

(57) ABSTRACT

Compositions derived from traditional Chinese herbal medicines, medicinal plants and extracts thereof, are provided for the prevention and treatment of cancers especially cancer of the lung, esophagus, stomach, oral cavity and prostate as well as for treating Helicobacter pylori infection. The compositions of the invention are obtained through specific techniques and have demonstrated efficacy for chemoprevention of cancers of the lung, esophagus, stomach, and the oral cavity as well as exhibiting in vitro activity against prostate cancer and leukemic cell lines. The compositions are useful as adjuncts to conventional surgery or radiotherapy treatments in patients with esophageal cancer. The compositions are effective as a treatment for H. pylori infection. Currently, there is no effective chemopreventive agent for these cancers. The current invention addresses a special need in cancer control. Preferred compositions of the invention contain the herbal ingredients Sophora tonkinensis, Polygonum bistorta, Prunella vulgaris, Sonchus brachyotus, Dictamnus dasycarpus Turcz, and Dioscorea bulbifera.

The present invention relates to compositions comprising herbs derived from traditional Chinese medicine and their use as chemopreventive and therapeutic agents. Processes for manufacturing and formulating the compositions are also described.

### BACKGROUND OF THE INVENTION

For the past twenty-five years there has been significant progress in the field of cancer research; however, in spite of these positive results, the mortality rate for the most common cancers still remains high. Indeed, the goal of the National Cancer Institute of a fifty percent reduction in overall cancer mortality by the year 2000 has not been met.

The term “cancer” is a general one referring to more than 100 forms of the disease which may manifest itself in almost every tissue type of the body. Of the myriad forms of cancer, lung cancer is the most common cause of death worldwide, followed by stomach cancer. Other common forms of cancer include cancers of the colon, rectum, breast, prostate, mouth and esophagus.

Lung cancer imposes an enormous burden on health care. The World Health Report 2000 estimates that lung cancer has resulted in 860,000 deaths among men and 333,000 deaths among women per year, and it is the leading cause of cancer deaths worldwide. In the United States and Canada, more people are dying from lung cancer than from breast cancer, colorectal cancer and prostate cancer combined. In addition, the incidence of lung cancer in women is rising at an average annual rate of 7% which is the most rapid rate of increase for any cancer. The most dominant cause of lung cancer is tobacco use, but occupational and environmental exposure to various other carcinogenic substances can also influence disease development. In long-term smokers, the risk of lung cancer never returns to the “baseline” level of a never-smoker, even years after smoking cessation. With a large reservoir (100 million in the United States and Canada alone) of current and former smokers, who are at risk, lung cancer will continue to be a major health problem for at least several more decades even if current efforts to curb tobacco smoking were successful. The overall five-year survival rate of lung cancer is less than 15%. Despite advances in modern medicine, the survival rate has not improved substantially over the last two decades. A different approach is therefore needed to control lung cancer.

Prostate cancer is the most commonly diagnosed male malignancy in the United States and the second leading cause of cancer death. It is estimated that in the year 2000, there will be 180,400 new cases and 31,900 deaths caused by prostate cancer. Although prostate cancer responds effectively to orchitectomy or antiandrogen therapy when detected at an early stage, over time, the residual androgen-insensitive cells recolonize, expand, and ultimately establish a hormone-resistant state that often results in fatality. It would be useful, therefore, to have a cancer treatment which could prevent the proliferation of prostate cancer and maintain it in a dormant state.

The incidence of gastric cancer has fallen in most countries but it is still the most common form of cancer in many countries of East Asia, including China. Globally, gastric cancer is the second most frequent cause of cancer death and it is estimated that in 1990, there were almost 800,000 new cases and about 630,000 deaths Similarly, esophageal cancer, the eighth most common cancer worldwide and responsible for 316,000 new cases and 286,000 deaths in 1990, is also very common in China and other Asian countries. Both of these cancers, and especially esophageal cancer, have low survival rates and thus it would be beneficial to have an alternative treatment approach for these types of cancers.

Traditionally, the focus of cancer research has been in developing therapies and treatments for patients already afflicted with the disease. However, over the last few decades, new insights into the development of cancer as a disease have been gained. It is now understood that cancer is not the result of a single initiation event but of a gradual, multi-step process characterized by a period of several years between the initiation event and the onset of invasive or metastatic disease. In general, the process of carcinogenesis can be divided into three phases: initiation, promotion, and progression. In initiation, a fixed genetic mutation results from the interaction of a carcinogen with DNA. The extent of the molecular change depends on a number of factors including the nature of the carcinogen, the rate and type of carcinogenic metabolism and the response of the DNA repair systems. The next phase, promotion, may occur over extended periods of time and is characterized by the proliferation of the altered cells. This phase may be affected by agents that alter growth rates. During the final phase, progression, genetic and phenotypic changes occur which ultimately cause the development of premalignant lesions into invasive cancer.

The multi-step nature of carcinogenesis suggests the possibility of intervention at a precancerous state. This is the basis for chemoprevention, which refers to the use of natural or synthetic agents to prevent the development of cancer, either by blocking the DNA damage that initiates the carcinogenesis process or by arresting/regressing existing pre-malignant lesions.

Since the mid-1950s, research has been directed at finding compounds with potential chemopreventive properties. The search for these agents has demonstrated a unique challenge. Chemopreventive agents must have low toxicity and be relatively free of side effects because they are intended for administration to healthy people over long periods of time. This is in direct contrast to chemotherapy drugs, such as cis platinum or paclitaxel (Taxol™), which are used as chemotherapeutic agents to treat people already afflicted with cancer. Chemotherapeutic agents are chosen for their ability to kill tumor cells but because they are also toxic to healthy cells, they usually cause harmful side effects.

One of the major sources of potential chemopreventive agents is plants. For example, consumption of cruciferous vegetables, such as broccoli, cauliflower and cabbage is associated with a lower risk of various cancers. Fruits and vegetables contain a number of potentially active chemopreventive compounds, such as carotenoids, dithiolthiones and isothiocyanates. They are capable of inhibiting the development of tumors of the lungs, colon, mammary glands and bladder in laboratory animals.

Three proof-of-principle clinical trials suggest that chemoprevention might be an effective strategy to control lung cancer. A study by Hong and co-workers in the United States showed that in patients with cured head and neck cancer, high dose 13-cis-retinoic acid for 12 months was more effective than placebo in preventing second primary cancers in the upper aerodigestive tract. However, 13-cis-retinoic acid at this dosage carries unacceptable toxicity for use in the general population. Another study by Pastorino and co-workers in Europe showed that retinol palmitate in a dose of 300,000 Units per day for 12 months was more effective than placebo in preventing second primary lung cancer. A third study in China found that daily doses of a combination of vitamins and minerals consisting of beta-carotene, vitamin E and selenium resulted in a 21% decrease in stomach cancer deaths in high-risk people in China. However, subsequent phase III clinical trials using beta-carotene or retinal (the Alpha-Tocopherol, Beta-Carotene Trial, the Beta-Carotene and Retinol Efficacy Trial, and the EUROSCAN study) failed to show a reduction in lung cancer incidence in high-risk individuals, such as heavy smokers with or without exposure to asbestos, compared to placebo. In fact, the use of beta-carotene in those who continued to smoke during the study was found to increase the risk of lung cancer. Several reasons were postulated to explain why chemopreventive treatment with retinoids was ineffective or even harmful in active smokers. There may be adverse interactions between tobacco carcinogens and the chemopreventive agent. Beta-carotene, for example, is a pro-oxidant at high arterial oxygen tension. It can enhance conversion of benzo[A]pyrene to the ultimate carcinogen as well as inducing cytochrome P450. Another reason for the lack of effect in active smokers is that ongoing tobacco carcinogen exposure may counteract the effect of the chemopreventive agent.

A number of chemopreventive agents are currently under clinical investigation. Examples of these include fenretinide, selenium, inhaled budesonide, COX-2 inhibitors, farnesyl transferase inhibitors, lipoxygenase inhibitors, EGF-kinase inhibitors and green tea. Although promising, it remains to be shown if these agents can be shown to be useful in ongoing clinical trials.

The best example to-date that chemoprevention can prevent cancer is Tamoxifen. Tamoxifen is an estrogen antagonist. In women at high risk of breast cancer, the Breast Cancer Prevention Trial showed a 49% decrease in invasive cancer and a 50% decrease in non-invasive breast cancer with Tamoxifen versus placebo (J Natl Cancer Inst 1998; 90:1371-88). In addition, there was a decrease in the incidence of fractures due to osteoporosis. However, there was a slight increase in the risk of endometrial cancer, deep venous thrombosis and pulmonary embolism. More selective estrogen-receptor modulators such as Raloxifene, are being tested against Tamoxifen to determine if these agents may have similar chemopreventive effect but fewer side effects than Tamoxifen.

A variety of Chinese herbs have been used for centuries to treat different diseases. The great majority of these are empirical, open, non-randomized studies without placebo control groups. Although some of these herbs have been used to treat patients with cancer, they are not considered to be disease specific. Rather, they are used for “dispersing heat, detoxification, improving stasis and removing mass”. Herbs such as Sophora tonkinensis, Polygonum bistorta, Prunella vulgaris, Sonchus brachyotus, Dictamnus dasycarpus Turcz, and Dioscorea bulbifera are known to have properties that may be useful for the prevention and treatment of cancers. One such composition is known as ZSP or Zeng Sheng Ping; however, the exact formulation of the composition is not known. In traditional Chinese herbal medicine, it is common practice to substitute selected herbs with other herbs depending upon the symptoms of an individual patient and/or availability of certain herbs locally. The need has therefore arisen for the development of compositions comprising well-defined mixtures of purified herbs for specific indications.

### SUMMARY OF THE INVENTION

Thus, according to the present invention there is provided a composition comprising herbs derived from traditional Chinese medicine.

Thus, according to this embodiment of the invention, there is provided a composition comprising a mixture of at least three different herbs selected from the group consisting of: Herb A, Herb B, Herb C, Herb D and Herb E, wherein Herb A is selected from the group consisting of:

Sophora tonkinensis (Sophora subprostrata)

Belamcanda chinensis (L) DC

Scrophularia ningpoensis Hemsl.

Isatis tinctoria L.

Isatis indigotica Fort. and

Baphicacanthus cusia Bremek;

Herb B is selected from the group consisting of:

Polygonum bistorta

Polygonum lapidosum Kitag.

Polygonum viviparum L.

Polygonum manshuriense V. Patr.

Polygonum alopecuroides Turcz.

Polygonum sphaerostachyum Meissn.

Andrographis paniculata (Burnf.) Nees

Taraxacum mongolicum Hand. and

Chrysanthemun indicum L;.

Herb C is selected from the group consisting of:

Prunella vulgaris

Artemissia capillaris Thunb.

Gardenia jasminoides Ellis.

Rosa rugosa Thunb. and

Lophatherum gracile Brongn.;

Herb D is selected from the group consisting of:

Sonchus brachyotus

Patrinia scabiosaefolia Fisch.

Patrinia villosa Juss.

Sonchus arvensis L.

Thlaspi arvense L.

Portulaca oleracea L. and

Pulsatilla chinensis Reg. and

Herb E is selected from the group consisting of:

Dictamnus dasycarpus

Kochia scoparia (L.) Schard.

Sophora flavescens Ait. and

Heydyotis diffusa (Willd.) Roxb.

In one example of this embodiment, the composition also comprises an herb from Herb F, which is selected from the group consisting of:

Dioscorea bulbifera

Panax notoginseng (Burk) F. H. Chen.

Bletilla striata (Thunb.) Reichb.f.

Nelumbo nucifera Gaertn.

Polygonum bistorta L.

Cephalanoplos segetum (Boge) Kitam.

Cirsium japonicum DC

Sophora japonica L.

Typha angustifolia L. and

Rubia cordifolia L.

Further according to the present invention there is provided a use of said composition as chemopreventive and therapeutic agents.

In one example of this further embodiment of the invention, there is provided a method of preventing or treating cancer by administering to a patient in need thereof an effective amount of a composition as defined above.

In a further example of this embodiment of the invention, there is provided a method of treating Helicobacter pylori infection by administering to a patient in need thereof an effective amount of a composition as defined above.

In yet a further example of this embodiment of the invention, there is provided a method of treating or reducing the reoccurrence of cancer by administering to a patient in need thereof an effective amount of a composition as defined above, as adjuncts to conventional surgery or radiotherapy treatments.

In yet a further embodiment of the present invention there is provided a process for the manufacture and formulation the compositions of the present invention.

In one example of this embodiment, there is provided a method of preparing the composition as defined above comprising the steps of: subjected the herbs to an aqueous extraction, optionally filtering the extract to remove large particles, drying the aqueous extract; and formulating the dried extract into a form suitable for administration.

### BRIEF DESCRIPTION OF THE DRAWINGS

These and other features of the invention will become more apparent from the following description in which reference is made to the appended drawings wherein:

FIG. 1 shows the HPLC profiles of herbal extracts of one of the embodiments of the present invention. Three aliquots of each herbal component were sampled in order to compare the within-batch consistency of the six herbal extracts A-F monitored at 330 nm. FIG. 1A is the profile of Sophora tonkinensis, FIG. 1B is the profile of Polygonum bistorta, FIG. 1C is the profile of Prunella vulgaris, FIG. 1D is the profile of Sonchus brachyotus, FIG. 1E is the profile of Dictamnus dasycarpus Turcz, and FIG. 1F is the profile of Dioscorea bulbifer. The HPLC profiles demonstrate that each botanical extract has a unique HPLC profile with specific peaks and show no significant differences within the batch of botanicals.

FIG. 2 shows the HPLC profiles of herbal extracts of FIG. 1, overlaid, with absorbance monitored at 330 nm (FIG. 2A), 260 nm (FIG. 2B) and 203 nm (FIG. 2C).

FIG. 3 shows the HPLC profiles of two herbal compositions, ACAPHA 1 and ACAPHA 2, comprising a combination of herbs of FIG. 1. FIG. 3A shows the batch-to-batch-consistency profiles of four batches of ACAPHA 1 monitored at 330 nm. FIG. 3B shows the batch-to-batch-consistency of three batches of ACAPHA 2 monitored at 330 nm. Although there are minor differences in the batch to batch profiles, overall they show no significant differences within the two different ACAPHA compositions.

FIG. 4 compares the HPLC profiles of three herbal compositions, ACAPHA 1, ACAPHA 2 and ACAPHA 3, comprising a combination of herbs of FIG. 1 monitored at 330 nm (FIG. 4A), 260 nm (FIG. 4B) and 203 nm (FIG. 4C). These HPLC profiles demonstrate that each ACAPHA composition has a unique profile with peak specificity. For example at 330 nm differences in peak sizes of the three ACAPHA formulations can be seen at retention time 4, 9, 12, 14 and 15 minutes.

FIG. 5 shows the mass spec profiles of herbs Sophora tonkinensis (FIG. 5A) and Polygonum bistorta (FIG. 5B).

FIG. 6 shows the mass spec profiles of herbs Prunella vulgaris (FIG. 6A) and Sonchus brachyotus (FIG. 6B).

FIG. 7 shows the mass spec profiles of herbs Dictamnus dasycarpus (FIG. 7A) and Dioscorea bulbifera (FIG. 7B).

FIG. 8 shows a mass spec profile of ACAPHA 1 herbal preparation of the invention. (Hewlett Packard 5989B Mass Spec Engine, SPLSCO51 Method, DB-512mX.2mX.33u column were used).

FIGS. 9-12: Each figure shows the mass spectrometry (MS) profiles of two chemicals detected in ACAPHA 1. The top panel shows the MS profile of ACAPHA 1 and for comparison the bottom panel shows the MS profile of the specific chemical from the MS library.

FIG. 9 shows the presence by MS of diosgenin (FIG. 9A) and 1-naphthalenecarbonitrile (FIG. 9B) in ACAPHA 1. With the same procedure diosgenin and 1-naphthalenecarbonitrile were detected in herbs Polygonum bistorta, Prunella vulgaris, Sonchus brachyotus, Dictamnus dasycarpus and Dioscorea bulbifera.

FIG. 10 shows the presence by MS of hexadecanoic acid (FIG. 10A) and beta-sitosterol (FIG. 10B) in ACAPHA 1. With the same procedure hexadecanoic acid was detected in herbs Sophora tonkinensis, Polygonum bistorta, Prunella vulgaris, Sonchus brachyotus and Dictamnus dasycarpus and, beta-sitosterol was detected in herbs Sophora tonkinensis, Sonchus brachyotus, Dictamnus dasycarpus and Dioscorea bulbifera.

FIG. 11 shows the presence by MS of campesterol (FIG. 11A) and stigmasterol (FIG. 11B) in ACAPHA 1. With the same procedure campesterol was detected in herbs Sonchus brachyotus, Dictamnus dasycarpus and Dioscorea bulbifera and, stigmasterol was detected in herbs Sophora tonkinensis, Dictamnus dasycarpus and Dioscorea bulbifera.

FIG. 12 shows the presence by MS of acacetin (FIG. 12A) and dictamnine (FIG. 12B) in ACAPHA 1. With the same procedure acacetin was detected in herb Sophora tonkinensis and, dictamnine was detected in herb Dictamnus dasycarpus.

FIG. 13 shows the overlapping chromatograms acquired using LC/MS. These chromatograms obtained from three samples ACAPHA 1 Lots 1, 2 and 3 demonstrate almost a perfect match between all three samples suggesting they are identical with respect to their chemistry.

FIG. 14 shows the age (FIG. 14A) and gender (FIG. 14B) distribution of patients in the ACAPHA 2 Phase III Clinical Trial of Chemoprevention in Patients with Esophageal Dysplasia (n=149 in placebo group; n=300 in ACAPHA 2 treatment group).

FIG. 15 is a bar graph showing changes in clinical symptoms after 6 months of ACAPHA 2 treatment (n=149 in placebo group; n=300 in ACAPHA 2 treatment group).

FIG. 16 shows changes in staging of esophageal dysplasia by endoscopy after 6 months of ACAPHA 2 treatment (n=149 in placebo group; n=300 in ACAPHA 2 treatment group).

FIG. 17 shows changes in staging of esophageal condition by biopsy histopathology grade after 6 months of ACAPHA 2 treatment (n=149 in placebo group; n=300 in ACAPHA 2 treatment group).

FIG. 18 is a plot comparing the survival rates of patients treated with ACAPHA 1 as an adjunct therapy to radiotherapy compared to radiotherapy alone in patients with esophageal cancer.

FIG. 19 is a plot comparing the survival rates of patients treated with ACAPHA 1 as an adjunct therapy to surgery compared to surgery alone in patients with esophageal cancer.

FIG. 20 is a bar graph comparing the survival rates of patients treated with ACAPHA 1 as an adjunct to conventional surgery and radiotherapy 12, 24 and 36 months post-treatment.

FIG. 21 is a bar graph showing the clinical response of individuals having bronchial dysplasia to treatment with ACAPHA 1 and other putative chemopreventive agents six months post-treatment analyzed using site specific analysis. The bars represent complete regression of dysplasia (CR), no response (NR) and progression of disease (PD)/or appearance of new dysplastic lesions under treatment. Retinol=Vitamin A; Sialor=Anetholtrithione; Pulmicort=Inhaled Budesonide.

FIG. 22 is a bar graph of the data of FIG. 21 analyzed using Person specific analysis.

FIG. 23 is a bar graph showing the clinical response of individuals having bronchial dysplasia to treatment with ACAPHA 1 six months post-treatment and six months off ACAPHA 1 analyzed using site specific analysis.

FIG. 24 is a bar graph of the data of FIG. 23 analyzed using person specific analysis.

FIG. 25 is a bar graph showing the growth suppressive effect of 72 hours of treatment of 3 μg/ml of ACAPHA 1 on androgen responsive LNCaP and androgen-refractory JCA-1, PC-3 and DU-145 prostate cancer cells.

FIG. 26 shows the effect of concentration (A, assessed after 72 hours of treatment) and duration of treatment (B) of ACAPHA 1 on androgen refractory JCA-1 prostate cancer cells in vitro.

FIG. 27 shows the comparison of effects of ACAPHA 1 (A1), ACAPHA 2 (A2), and ACAPHA 3 (A3) on colony formation using androgen-refractory (DU-145, JCA-1 and PC-3) and androgen-responsive (LNCaP) cells.

FIG. 28 compares the effects of 72 hours of treatment with various concentrations of ACAPHA 1 (A1), ACAPHA 2 (A2) and ACAPHA 3 (A3), respectively, on changes in the distribution of cell cycle phase progression in the androgen-refractory human prostate JCA-1 cell line.

FIG. 29 compares effects of 72 hours of treatment with various concentrations of ACAPHA 1 (A1), ACAPHA 2 (A2) and ACAPHA 3 (A3), respectively on changes in the distribution of cell cycle phase progression in the androgen-responsive human prostate LNCaP cells.

FIG. 30 shows the effects of various concentrations of ACAPHA 1 (A1), ACAPHA 2 (A2), and ACAPHA 3 (A3), respectively, on time-dependent increase in secreted PSA in media of androgen-responsive human prostate LNCaP cells. Cells were treated with ACAPHA for 1, 2, or 3 days. PSA in the culture media was determined using the TANDEM E kit from Beckman Coulter.

FIG. 31 shows the Western blot analysis of effects of various concentrations of ACAPHA 1 (A1), ACAPHA 2 (A2), and ACAPHA 3 (A3), respectively, on expression of secreted PSA in media of androgen-responsive human prostate LNCAP cells. Cells were treated with ACAPHA for 3 days. PSA in the culture media was determined using PSA cognate antibody and visualized by color reaction.

### DETAILED DESCRIPTION OF PREFERRED EMBODIMENT

The present invention relates to compositions comprising herbs derived from traditional Chinese medicine and their use as chemopreventive and therapeutic agents. Processes for manufacturing and formulating the compositions are also described.

In one embodiment of the present invention the composition comprises a mixture of at least three different herbs, known hereinafter as Herb A, Herb B, Herb C, Herb D and Herb E.

According to the present invention Herb A is selected from the group consisting of:

Sophora tonkinensis (Sophora subprostrata)

Belamcanda chinensis (L) DC

Scrophularia ningpoensis Hemsl.

Isatis tinctoria L.

Isatis indigotica Fort. and

Baphicacanthus cusia Bremek

According to the present invention Herb B is selected from the group consisting of:

Polygonum bistorta

Polygonum lapidosum Kitag.

Polygonum viviparum L.

Polygonum manshuriense V. Patr.

Polygonum alopecuroides Turcz.

Polygonum sphaerostachyum Meissn.

Andrographis panicutata (Burm.f.) Nees.

Taraxacum mongolicum Hand. and

Chrysanthemun indicum L.

According to the present invention Herb C is selected from the group consisting of:

Prunella vulgaris

Artemissia capillaris Thunb.

Gardenia jasminoides Ellis.

Rosa rugosa Thunb. and

Lophatherum gracile Brongn.

According to the present invention Herb D is selected from the group consisting of:

Sonchus brachyotus

Patrinia scabiosaefolia Fisch.

Patrinia villosa Juss.

Sonchus arvensis L.

Thlaspi arvense L.

Portulaca oleracea L. and

Pulsatilla chinensis Reg.

According to the present invention Herb E is selected from the group consisting of:

Dictamnus dasycarpus

Kochia scoparia (L.) Schard.

Sophora flavescens Ait. and

Heydyotis diffusa (Willd.) Roxb.

When, according to the present invention, the composition comprises three different herbs, one of which is selected from each of Herb A, Herb B, Herb C, Herb D or Herb E, each of the three herbs is present in an amount from about 9% to about 57%. In a further example of this embodiment, the three herbs are each present in an amount from about 15% to 50%. In a further example of this embodiment, each of the three herbs are present in an amount from 25% to 40%. In a further example of this embodiment, each of the three herbs are present in an amount of about 33%.

When the composition comprises four different herbs, one of which is selected from each of Herb A, Herb B, Herb C, Herb D or Herb E, each of the four herbs are present in an amount from about 6% to about 43%. In a further example of this embodiment, each of the four herbs are present in an amount from about 15% to about 35%. In a further example of this embodiment, each of the four herbs are present in an amount of about 25%.

In a further example of the present invention, when the composition comprises four herbs, three of which are selected from the group consisting of Herb A, Herb B, Herb C, Herb D and the fourth herb is selected from the group consisting of Herb E. The composition of the first three herbs are each present in an amount from about 7% to about 57% and the fourth herb is present in an amount from about 3.5% to about 28.5%. In a further example of this embodiment, each of the first three herbs are present in an amount from about 15% to about 48% and the fourth herb is present in ! an amount from about 7.5% to about 24%. In a further example of this embodiment, the first three herbs are present in a composition in an amount of about 20% to about 43%, and the fourth herb is present in an amount from about 10% to about 21.5%.

In a further example of this embodiment, the first three herbs are present in an amount of about 28% and the fourth herb is present in an amount of about 14%.

In yet a further embodiment of this invention, the composition comprises five different herbs, one of which is each selected from Herb A, Herb B, Herb C, Herb D or Herb E, each of the five herbs are present in an amount from about 5% to about 35%. In a further example of this embodiment, each of the five herbs are present in an amount from about 10% to about 30%. In yet a further example of this embodiment, each of the five herbs are present in an amount from about 15% to about 25%. In a further example of this embodiment, each of the five herbs are present in an amount of about 20%.

In yet a further embodiment of this invention, the first four herbs are selected one from each of the group Herb A, Herb B, Herb C and Herb D whereas the fifth herb is selected from Herb E and the first four herbs are present in an amount from about 6% to about 38%, whereas the fifth herb is present at an amount from about 3% to about 19%. In a further example of this embodiment, the first four herbs are present in the composition at an amount from about 12% to about 32%, and the fifth herb is present in an amount from about 6% to about 16%. In yet a further example of this embodiment, the first four herbs are present in an amount from about 18% to about 26% and the fifth herb is present in an amount from about 9% to about 13%. In yet a further example of this embodiment, the first four herbs are present at an amount of about 22%, whereas the fifth herb is present at an amount of about 11%.

In one example of this embodiment the composition comprises a mixture of the following herb: Sophora tonkinensis, Polygonum bistorta and Prunella vulgaris.

In one further example of this embodiment, the composition comprises a mixture of the following herbs: Sophora tonkinensis, Polygonum bistorta, Prunella vulgaris and Dictamnus dasycarpus.

In one example of this embodiment the composition comprises a mixture of the following herbs: Sophora tonkinensis, Polygonum bistorta, Prunella vulgaris, Sonchus brachyotus and Dictamnus dasycarpus Turcz.

In a further embodiment of the present invention the composition comprises a sixth herb, Herb F. In this embodiment Herb F is selected from the group consisting of:

Dioscorea bulbifera

Panax notoginseng (Burk) F. H. Chen.

Bletilla striata (Thunb.) Reichb.f.

Nelumbo nucifera Gaertn.

Polygonum bistorta L.

Cephalanoplos segetum (Boge) Kitam.

Cirsium japonicum DC.

Sophora japonica L.

Typha angustifolia L. and

Rubia cordifolia L.

In one example of this embodiment, the first five herbs are selected from the group consisting of Herb A, Herb B, Herb C, Herb D and Herb E, and the sixth herb is selected from Herb F and the first five herbs are present in an amount from about 5% to about 31% and the sixth herb is present in an amount from about 2.5% to about 17.5%. In a further example of this embodiment, each of the first five herbs are present in an amount from about 10% to about 26%, and the sixth herb is present in an amount from about 5% to about 15%. In a further example of this embodiment, the first five herbs are each present in an amount from about 15% to about 21% and the sixth herb is present in an amount from about 7.5% to about 12.5%. In a further example of this embodiment, the first five herbs are present in an amount of about 18% and the sixth herb is present in an amount of about 10%.

In yet a further example of this embodiment, the first four herbs are selected from the group of Herb A, Herb B, Herb C or Herb D. The fifth herb is selected from Herb E and the sixth herb is selected from Herb F, wherein the first three herbs are each present in an amount of about 5% to about 38%, the fifth herb is present in an amount of about 2.5% to about 19% and the sixth herb is present in an amount of about 1.75% to about 9.5%. In a further example of this embodiment, the first four herbs are present in an amount of about 10% to about 33%. The fifth herb is present in an amount of about 5% to about 16.5% and the sixth herb is present in an amount of about 3% to about 8.25%. In yet a further example of this embodiment, the first four herbs are present in an amount of about 15% to about 28%; herb five is present in an amount of about 75% to about 14.5% and the sixth herb is present in an amount of about 4.25% to about 7%. In yet a further example of this embodiment, the first four herbs are present in an amount of about 21%. The fifth herb is present in an amount of about 10.5% and the sixth herb is present in the amount of about 5.5%.

In one example of this embodiment the composition comprises a mixture of the following herbs: Sophora tonkinesis, Polygonum bistorta, Prunella vulgaris, Sonchus brachyotus, Dictamnus dasycarpus Turcz, and Dioscorea bulbifera.

The botanical, chemical and histological characteristics of the component herbs found in some embodiments of the present invention are described further below.

Sophora tonkinensis

Scientific Names:

Pharmaceutical name: Radix Sophorae Tonkinensis

Botanical name: Sophora tonkinensis Gapnep, also known as Sophora subprostrata

Synonyms and Common Names:

Guang Dou Gen

Shan- dou gen- (Mandarin)

Sanzukon (Japanese)

Santugún (Korean)

Vietnamese Sophora root (English)

Pigeon pea root

Mountain bean root

subprostrate

Botanical Family:

Leguminosae

Parts used:

Dried Roots

Definition of Drug:

Vietnamese Sophora Root is the dried root and rhizome of

Sophora tonkinensis Gapnep.

Chemical Constituents:

Matrine

Oxymatrine

Anagyrine

Methylcytisine

Sophoranone

Sophoradin

Sophoranochromente

Sophoradochromente

Genistein

Pterocarpine

Maackian

Trifolirhizin

Sitosterol

Hexadecanoic acid

Approximately 0.93% of the root of Sophora tonkinensis consists of alkaloids, including matrine, oxymatrine, anagyrine and methylcytisine. The root contains other non-alkaloidal substances, including sophoranone, sophoranochromene, sophoradin and daidzein (C15H₁₀O₄). Also present is 1-trifolirhizin (CH₂₂H₂₂O₁₀), a glucoside whose hydrolysed product is 1-maackinin (C₁₆H₁₂O₅). Other substances isolated from Sophora tonkinensis include pterocarpine (C,₇HI406) and 1-maackain. Also, some triterpenoidal saponins have been isolated from the root: these include sophorneoanodromone and sophorneoadochromone.

Botanical Characteristics:

General Distribution:

The herb's distribution in China is in Guangxi, Guizhou and Yunnan provinces. The elevation range in China is between 1000-2000 m. It is also distributed in Vietnam.

Macroscopic Description:

Rhizomes are irregularly nodiform showing remaining stem-bases at the top and several roots at the lower part. Roots are usually branched and long cylindrical. Its length is variable and its diameter is between 0.7-1.5 cm. The outer part is brown to dull brown with irregular longitudinal wrinkles and protruding transversal lenticels. The texture is hard and tough with brownish fractured surface in bark and pale yellow in wood. Its characteristic odour is similar to bean and its taste is very bitter.

Histology:

A transverse section demonstrates that the cork layer consists of up to ten or more layers of cells. In the cortex the first and second outer rows of cells contain prisms of calcium oxalate which create a discontinuous ring of crystal cells with lignified and thickened cell walls. Groups of fibres are distributed throughout both the cortex and phloem. The cambium is in a ring. Xylem is well developed, the rays are 1-8 cells wide; vessels are subrounded, mostly single and scattered or in groups of two or more, containing yellowish-brown contents; wood fibres are scattered in groups. Parenchymatous cells are filled with starch grains and a few cells contain prisms.

Polygonum bistorta

Polygonum bistorta contains a number of chemical compounds including polyphenols such as ellagic acid, tannins, phlobaphene, flavonoids and a trace of the anthraquinone emodin. Other chemical constituents include hexadecanoic acid, diosgenin, iodoquinol, and 1,4,2-dioxazole, 3-(4 chlorphenyl)-5-ph.

Scientific Names:

Pharmaceutical name: Rhizoma Polygonum Bistorta L.

Botanical name: Polygonum bistorta L.

Synonyms and Common Names:

Bistort

Dragonwort

Easter Giant

Adderwort

English Serpentary

Osterick, Passions

Patience Dock

Red Legs

Sweet Dock

Snakeweed

Quánshen (Chinese)

Botanical Name:

Polygonaceae

Parts used:

Dried rhizomes

Definition of Drug: Bistort root and rhizome is the subterranean part of Polygonum bistorta L.

Chemical Constituents:

Bistort contains polyphenols (including ellagic acid), tannins (15-20%), phlobaphene, flavonoids and a trace of the anthraquinone emodin.

The chemical constituents are listed as:

Hexadecanoic acid

Diosgenin

Iodoquinol

1,4,2dioxazole,3-(4 chlorphenyl)-5-ph

Botanical Characteristics:

General Distribution:

Polygonum bistorta is distributed in Europe, Northern and Western Asia; China, Japan, and North America. This perennial grows on slopes, in grassland and under trees; meadows and woodland, and prefers damp conditions.

Macroscopical Description:

Polygonum bistorta is a perennial herb, about 50-80 cm high. Rootstock is crooked, robust, woody and purplish-brown or black, as thick as the thumb or less. The stem is erect, very simple, slender and glabrous.

The basal leaves are clustered and long petiolate, oblong-lanceolate, 10-18 cm long by 2.5-5 cm wide. The apex is acute and the base is obtuse-rounded or truncate, sometimes cordate (heart-shaped) and coriaceous. Sheath of stipules are tube-like, membranous. Cauline leaf is usually linear or lanceolate, sessile or amplexicaul.

The flowers are reddish or white. Petals are present in numbers of 5 and are elliptic. There are 8 stamens and 3 stigmas. The nut is very small, trigonous, glossy and reddish-brown colored. The herb is odorless but tastes very bitter and harsh.

Histology:

The transverse section of the rhizoma is pink. The plant's surface when transversely fractured is brownish red and shows vascular bundles arranged in a circle of yellowish white dots.

Prunella vulgaris

The whole dried plant of Prunella vulgaris is used to produce the compositions of the present invention. The chemical constituents include oleanolic acid, ursolic acid, rutin, hyperoside, caffeic acid, Vitamins B1, C and K, tannin, polysaccharide prunelline, and triterpenes.

Scientific Names:

Pharmaceutical name: Spica Prunellae Vulgaris

Botanical name: Prunella vulgaris L. (Brunella vulgaris L.)

Synonyms and Common names:

Self heal, Heal all, selfheal spike, prunella,

“Summer withered herb” (English)

Kagos_(Japanese)

Hagoch'o (Korean)

Xiã kucao (Chinese)

Botanical Family:

Labiatae

Parts used:

Fruit spike

Definition of Drug:

The drug, common Selfheal Fruit-Spike, consists of the dried flowered fruit-spike of Prunella vulgaris L.

Chemical constituents:

Oleanolic acid

Ursolic acid

Rutin

Hyperoside

Caffeic acid

Vitamin B1

Vitamin C

Vitamin K

Tannin

Polysaccharide prunelline

Triterpenes (two), based on ursolic and

Oleanolic acid

Botanical Characteristics:

General Distribution:

Prunella vulgaris is distributed in China (Jiangsu, Anhhui, Zhejiang, Hunan) and Japan.

Macroscopic Description:

The herb is a common weed, a low sprawling perennial about 45 cm high, with a short rhizome, reproducing by seeds and short runners that root freely at the nodes. As with other members of the mint family, the stems are square and the leaves are opposite. The stems branch freely and are usually about one foot tall.

The lower leaves are petioled with the blades lanceolate to ovate. The upper leaves are sessile, subtending the flower cluster. The flower cluster is a close thick spike with three flowers in the axils of each rounded membranaceous bract.

The flowers are two-lipped, the upper lip hood-shaped, the lower lip shorter and three lobed. The blossoms are lavender to white The herb tastes bitter.

Sonchus brachyotus

Scientific name:

Pharmaceutical name: Herba cum Radice Patriniae

Botanical name: (in China) Sonchus brachyotus DC. and Sonhus spp., (in Japan) Patrinia villosa Juss.,(in Taiwan and Hong Kong) Thlaspi arvensis L.

Family name:

Valerianaceae (Japan)

Cruciferae (Taiwan and Hong Kong)

Compositae (China)

Synonyms/Common name:

Pinyin qu mai cai

Haishoso (Japanese)

P'aechangch'o (Korean)

Patrinia thlaspi (English)

Snow-thistle, Penny-cress, Patrinia,

Pai-chiang-Tsao, or Bai-Jiang-Cao

Parts used:

Dried whole plant

Definition of Drug:

Chemical constituents:

Thiaspi arvensis (seeds): sinigrin and myrosinde (enzyme)

S. brachyotus DC (plant): fatty oil, ceryl alcohol, invert sugar, choline, tartaric acid; (juice) oxydase, kautschuk, mannitol, L-inositol, alpha and beta-lactucerol Patrinia villosa (root and rhizome) loganin, viloside, morroniside; (fruit and stem) sinigrin Patrinia scabiosaefolia (root and rhizome) oleanolic acid, hederagenin, beta-sitosterol, D-glucoside, saponins (patrinoside A,C,C,D, and scabiosides A,B,C,D,E,F,G), ursolic acid

Botanical Characteristics:

General Distribution:

The China provincial distribution of Sonchus brachyotus DC is in Heilongjiang, Jilin, Nei Mongol, Hebei, Shanxi, Shaanxi and Shandong. It is also distributed in Japan, Mongoia and Russia.

Dictamnus dasycarpus Turcz

The chemical constituents of Dictamnus dasycarpus Turcz include the alkaloids dictaine (C12H9O2N), skimmianine (C14H13O4N), g-fragarine (C13H11O3N), preskinnianine, and isomaculosindine, limonin (C26H30O8), obakinone, fraxinellone, psoralen (C11H6O3), aurapten, bergapten, aponins and essential oils.

Scientific name:

Pharmaceutical name: Cortex Dictamni Dasycarpi Radicis

Botanical name: Dictamnus dasycarpus Turcz.

Family: Rutaceae

Synonyms/Common names:

Densefruit, Pittany Root-bark

Bai Xian Pi (Mandarin)

Hakusenpi (Japanese)

Paeksonp'i (Korean)

Cortex of Chinese Dittany Root (Eng.)

“White Fresh Bark”

Parts used:

Dried root bark

Definition of drug:

Dense fruit Pittany Root-bark is the dried root bark of Dictamnus dasycarpus Turcz. The root bark is stripped off and dried.

Chemical constituents:

Several substances have been isolated from the bark. These include alkaloids:

dictanine (C₁₂H₉O₂N)

skimmianine (C₁₄H₁₃O₄N)

g-fragarine (C₁₃H₁₁O₃N)

preskinnianine and isomaculosinidine

limonin (C₂₆H₃₀O₈) and obakinone

fraxinellone

psoralen (C₁₁H₆O₃)

aurapten and bergapten

and saponins and essential oils.

Botanical Characteristics:

General Distribution:

The herb is native to China (Manchuria, Hebei, Henan, Anhui, Jiangsu, and Hubei), and Korea.

Macroscopical Description:

The root bark is quilled, 5-15 cm long, 1-2 cm in diameter, 2-5 mm thick outer surface greyish-white or pale greyish-yellow, with fine longitudinal wrinkles and rootlet scars, frequently with protruding small granular dots.

The inner surface is whitish with fine longitudinal wrinkles, smooth, slightly fibrous. The texture is fragile, dusting on breaking, fracture uneven and somewhat lamellar. When the outer layer is peeled off, numerous small glitter spots are observed during exposure to light. Its odor is muttony, and its taste is slightly bitter. The herb also has cold property.

Dioscorea bulbifera

The chemical constituents of the dried tubers of Dioscorea bulbifera are diosgenin, diosbulins A, B, and C, iodine and vitamins A, B, and C. This herb also contains a small amount of saponins, including dioscorecin, dioscoretoxin, and tannin

Scientific name:

Pharmaceutical name: Tuber Dioscoreae Bulbiferae

Botanical name: Dioscorea bulbifera L

Family: Dioscoreaceae (625 species)

Synonyms/Common names:

Bulbil-bearing yam, Potato yam,

Aerial yam, Air potato,

“Yellow medicine”

Huáng ya\overline{o} zi (Chinese)

Oyakushi (Japanese)

Hwangyakcha (Korean)

Parts used:

Dried tubers

Definition of drug:

Chemical constituents:

Diosgenin

Diosbulins B, C

Iodine

Vitamin A

Vitamin B

Vitamin C

The herb contains a small amount of saponins, including dioscorecin, dioscoretoxin and tannin. Some bitter substances are present as are diosbulbine A, B, and C, and iodine.

Botanical Characteristics:

General Distribution:

The Dioscoreales are primarily a tropical group, with a few species in Europe and temperate America. Most are vines or climbing plants, but some are herbs as well. The Dioscoreales include 600 species of Dioscorea and about 35 species in other genera. The group is not especially large, but the genus Dioscorea is common in wet tropical regions around the world. In many countries, it is cultivated for its starchy tubers, sometimes called air potatoes or Chinese yams.

Macroscopic Description:

A common characteristic of most Dioscoreales is a thick, starchy underground stem called a tuber. The tuber provides an energy store for these plants to begin growth again when conditions are favorable. The tubers of Dioscorea are full of starch and nutrients, and so are boiled and eaten in many cultures. These provide the primary source of carbohydrates for about 70 million people in Africa and southern Asia.

The plants are propagated by cutting up the tubers, rather than from seed, as there are not usually many seeds produced. The Dioscoreales are monocots. The leaves have a reticulated venation reminiscent of the paleoherb dicots, and may be heart-shaped or highly lobed. For this reason, the Dioscoreales have, at times, been considered the most “primitive” of monocots, but though they may retain many characters found in early monocots, they now appear to be more closely related to the Lilliales.

The Dioscoreales may be divided into the following families:

1. Trichopodaceae—Trichopus zeylanicus

2. Dioscoreaceae—Air potatoes (625 species), mostly Dioscorea

3. Taccaceae—only Tacca, the “bat flower”, with 10 species.

Dioscorea bulbifera is covered with large handsome leaves. It has a winter dormant period when the stems die back to ground. After dormancy, the underground tubers give rise to stems which grow quickly, reaching up to 70 feet by the end of the growing season. The vine's stem is herbaceous, not woody. The stem is round, and not winged.

The large leaves are up to 8 inches long and heart-shaped (cordate). The leaf blade's basal lobes are rounded. Leaf veins radiate from a single point. The leaves have long stems (petioles), and are alternate on the stem.

Unisexual flowers appear from July to October. They are small, greenish and fragrant, hanging in relatively long clusters (panicles and spikes) up to 4 inches long. The fruit is a capsule of seeds, winged at the base. Dioscorea bulbifera plants produce “aerial tubers” that are attached closely to the stems where leaves attach (axil). These are greyish and somewhat irregular. Tubers also grow underground where they may be much larger, light yellow to yellowish-brown, and have a bitter taste.

It is important to ensure that the herbs which are used according to the present invention are selected such that they contain only acceptable levels of contaminants such as metals or pesticides. Various regions of China have been surveyed and it was found that the component herbs are preferably harvested from the Guangxi, Hunan, Liaoning, Anhui, Hebei and Jiangsu regions of China during the summer and autumn months and no pesticides are used. The plants are purchased dried and whole or parts of these herbs are used in manufacturing.

The general manufacturing scheme is as follows: the herbs are either subjected to an aqueous extraction, the aqueous extract is then filtered if necessary to remove large particles, the aqueous extract is then dried to a powder. Alternatively, it is possible to use the herbs directly by grinding to a powder. The powdered herbs are then used in the production of the therapeutic in a variety of forms for administration.

In one example of the present invention the manufacturing process is as follows:

Dictamnus dasycarpus Turcz (105 g) is ground into a powder form and set aside for later use. Sophora tonkinensis (420 g) is added to ten times its volume in water (approximately 5L) and then set to boil and decoct for 15 hours. The fluid is then removed and saved and another ten times volume of water is added. The mixture is boiled and decocted for another 1.5 hours. The, fluid is again removed and then combined with the first portion. The solution is filtered to remove large particles and then concentrated by heating at reduced pressure to S.G. of 1.30 to 1.35 (50° C.) (cream paste). The cream is dehydrated at a temperature under 60° C., pulverized and the Sophora tonkinensis powder is then set aside for later use.

Another portion of Dictamnus dasycarpus Turcz (105 g) is mixed with Polygonum bistorta (420g), Prunella vulgaris (420 g), Sonchus brachyotus (420 g) and Dioscorea bulbifera (100 g). The mixture is added to ten times its volume in water (approximately 15L), boiled and then decocted for 2 hours. The fluid is then removed and saved. Another ten times volume of water is added and the boiling and decocting process is repeated. The fluid is again removed and added to the first portion. The solution is filtered to remove large particles and then concentrated at reduced pressure to S.G. of 1.30 to 1.35 (50° C.) (clear cream paste). The cream paste is then mixed with the Dictamnus dasycarpus powder and the Sophora tonkinensis powder. The mixture is then dehydrated at temperatures under 80° C. The dried herbal extract is then ground to a powder. Appropriate amount of starch and a small amount of ethanol is added as binding material and 0.3 g tablets are formed. Sugar is then used to coat the tablets. The recipe produces about 1,000 tablets. Thus, in this example of the present invention the herbs listed above, as an example of Herb A, Herb B, Herb C, Herb D, Herb E and Herb F are mixed together in the following proportion: 5% to 38%: 5% to 38%: 5% to 38%: 5% to 38%: 5% to 38%: 2.5% to 19%: 1.75% to 9.5%. As previously discussed it is possible to make other formulations using any of the alternative herbs as listed above for each the Herb groups.

Thus one example of the present invention is a combination of Sophora tonkinensis, Polygonum bistorta, Prunella vulgaris, Sonchus brachyotus, Dictamnus dasycarpus Turcz, and Dioscorea bulbifera in an amount of about 21%, 21%, 21%, 21%, 10.5%, and 5.5%, respectively, and is referred to in the present application as ACAPHA 1 or ACAPHA-A1.

In a further embodiment of the present invention Herb F is reduced by about 75%. As will be shown in the Examples, certain patients taking ACAPHA 1 showed elevated liver enzymes. Thus, according to this embodiment of the present invention, the formulation comprises a lower concentration of the Herb F. In one example of this embodiment there is provided a composition comprising a combination of Sophora tonkinensis, Polygonum bistorta, Prunella vulgaris, Sonchus brachyotus, Dictamnus dasycarpus Turcz, and Dioscorea bulbifera in an amount of about 21.9%, 21.9%, 21.9%, 21.9%, 11.0%, 1.4% and is referred to in the present application as ACAPHA 2 or ACAPHA-A2.

In yet a further embodiment of the present invention the Herb F is eliminated entirely from the composition. Thus, according to this embodiment the composition comprises a combination of Herb A, Herb B, Herb C, Herb D and Herb E in an amount of from 6%-38%, 6%-38%, 6%-38%, 6%-38%, and 3%-19%, respectively. In one example of this embodiment there is provided a composition comprising a combination of Sophora tonkinensis, Polygonum bistorta, Prunella vulgaris, Sonchus brachyotus and Dictamnus dasycarpus Turcz in an amount of about 22%, 22%, 22%, 22%, and 11%, respectively and is referred to in the present application as ACAPHA 3 or ACAPHA-A3.

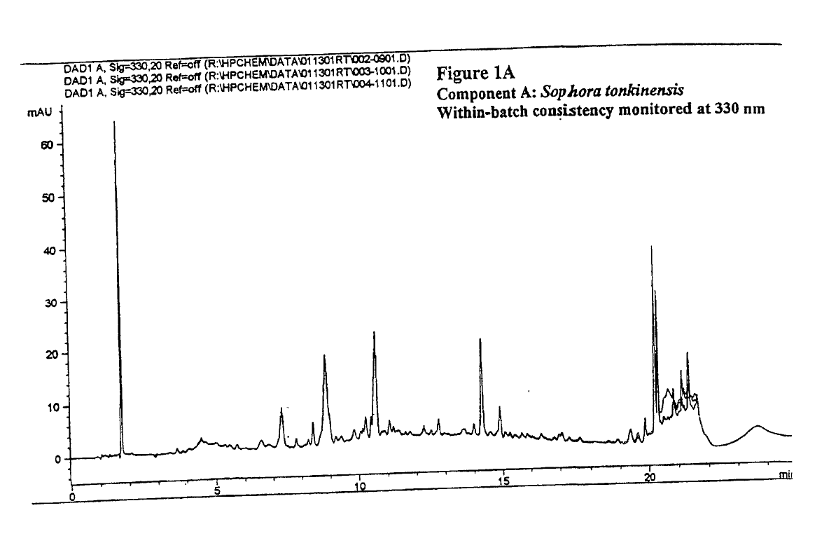

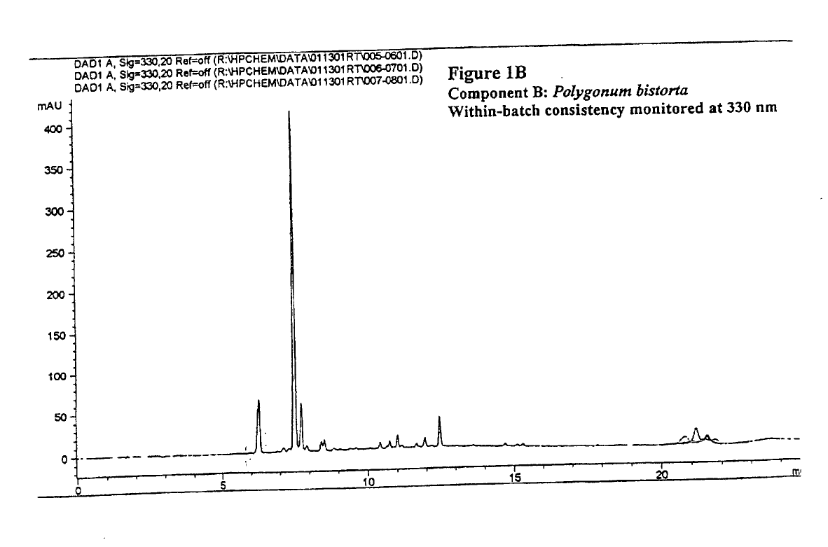

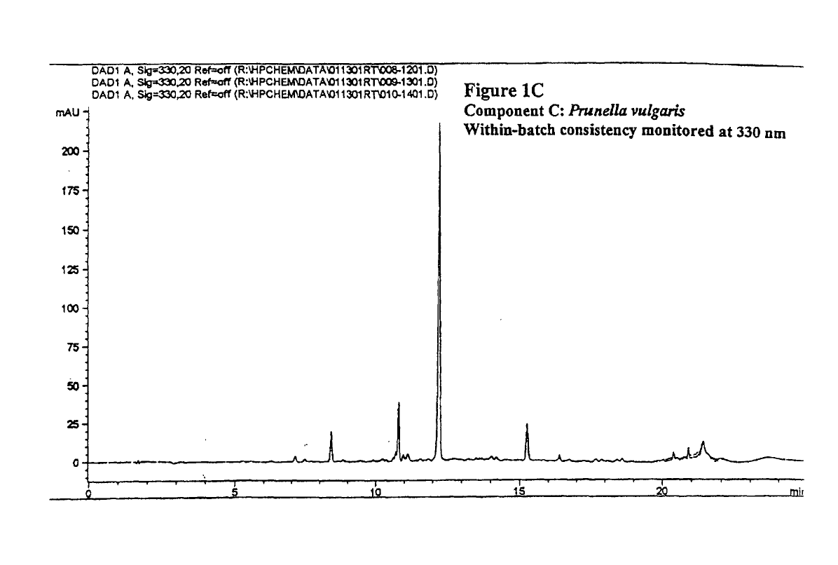

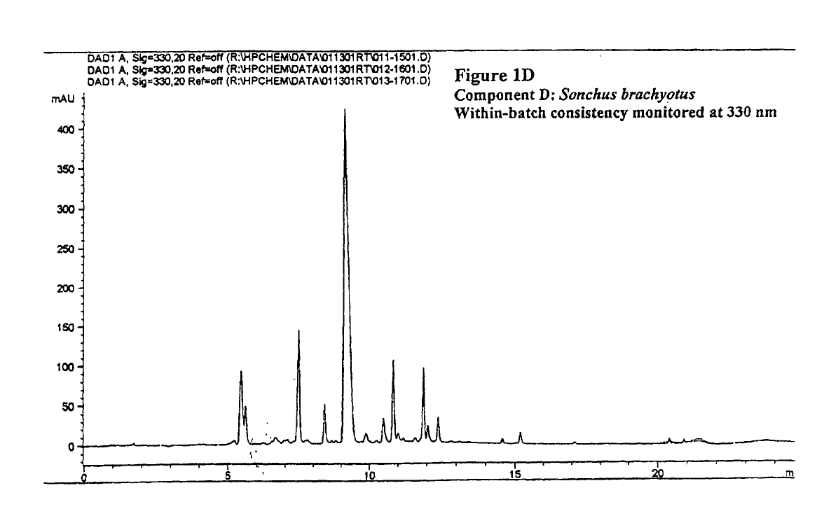

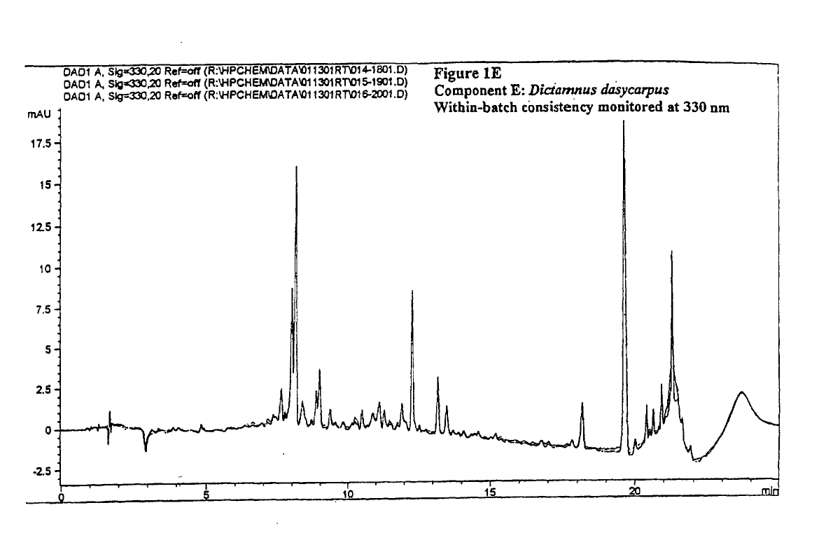

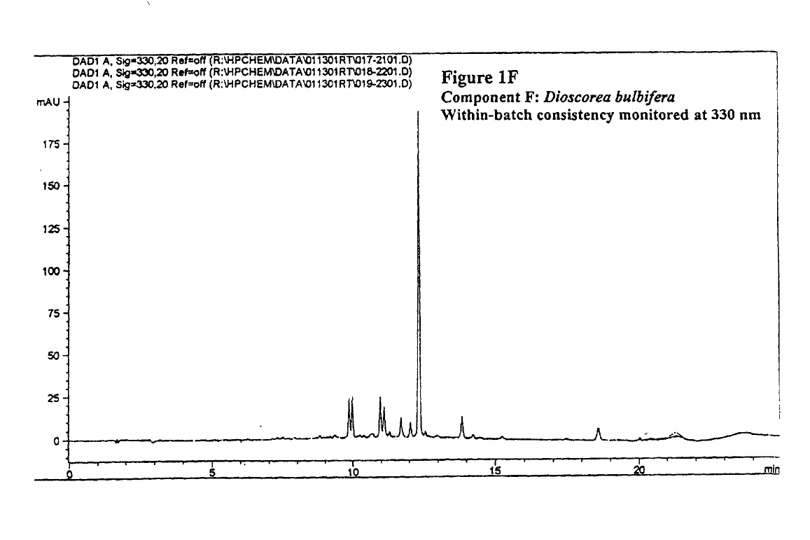

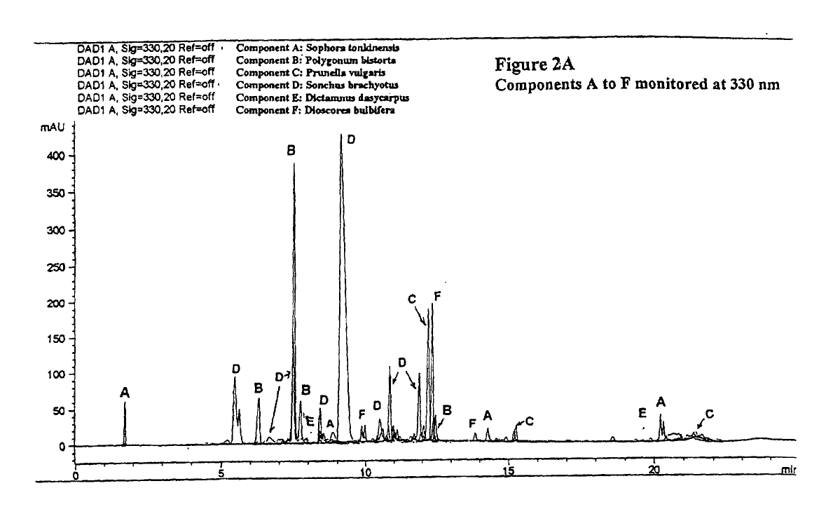

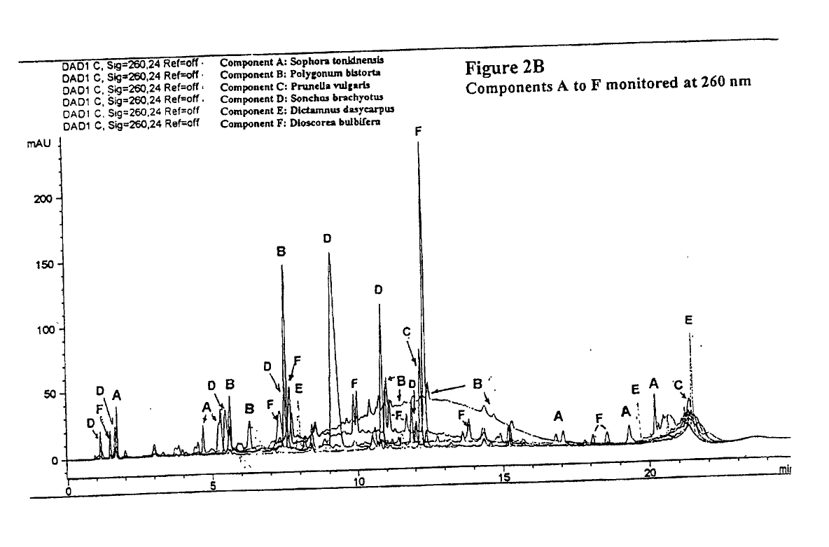

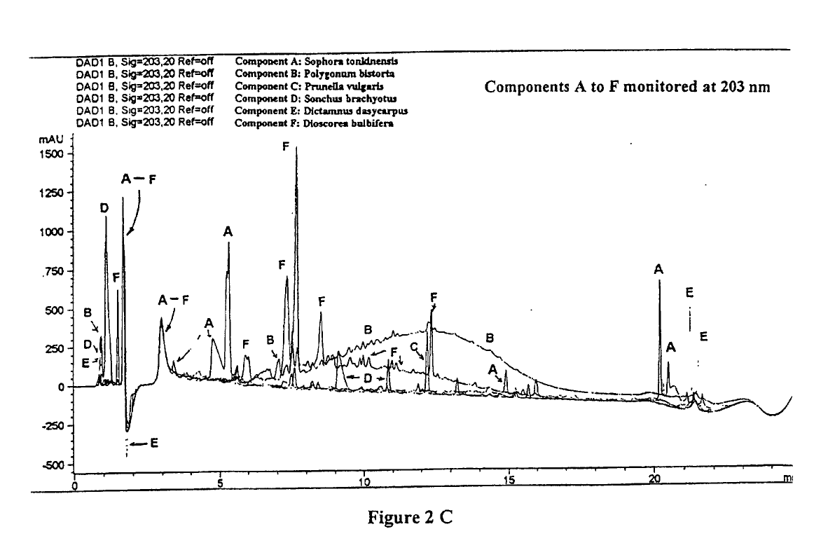

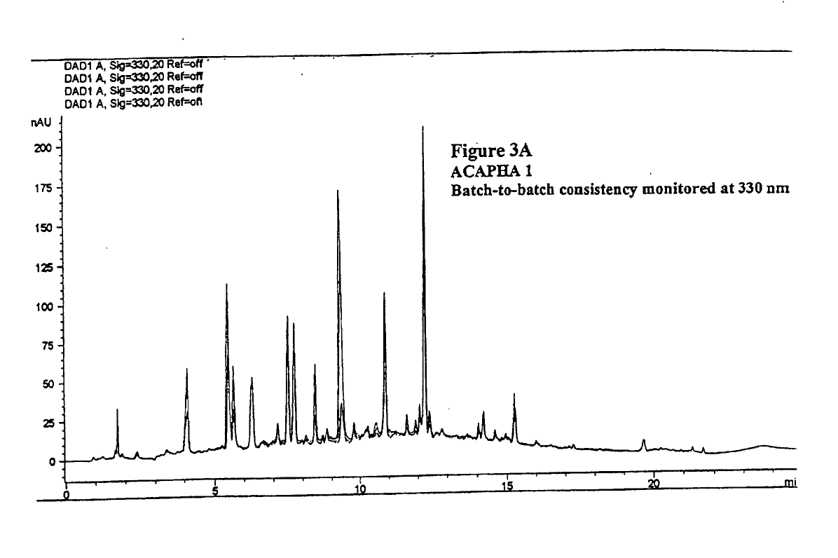

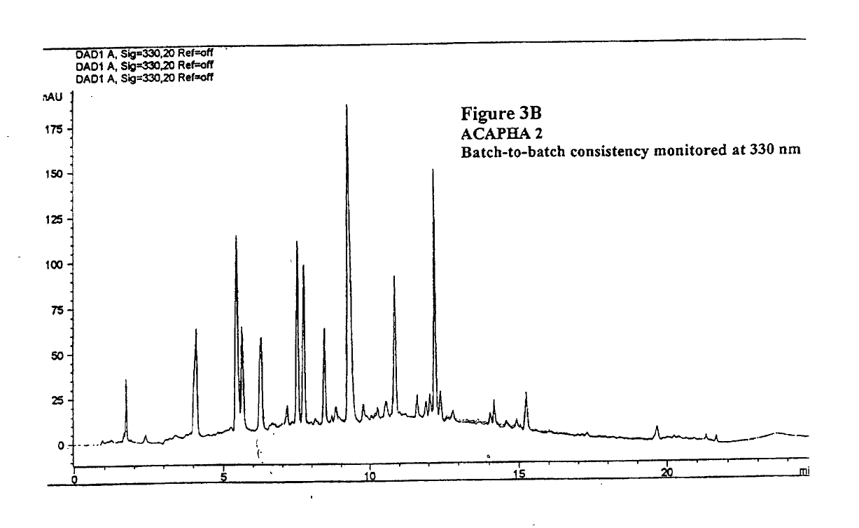

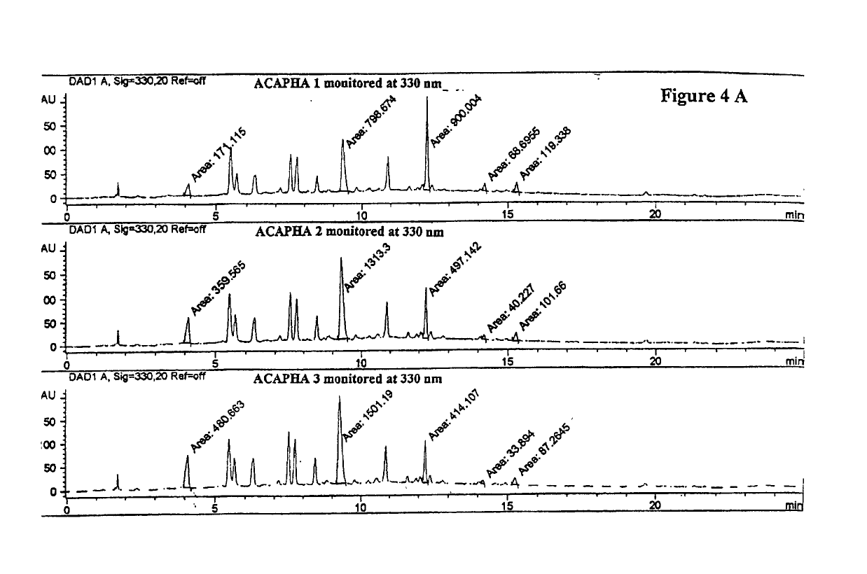

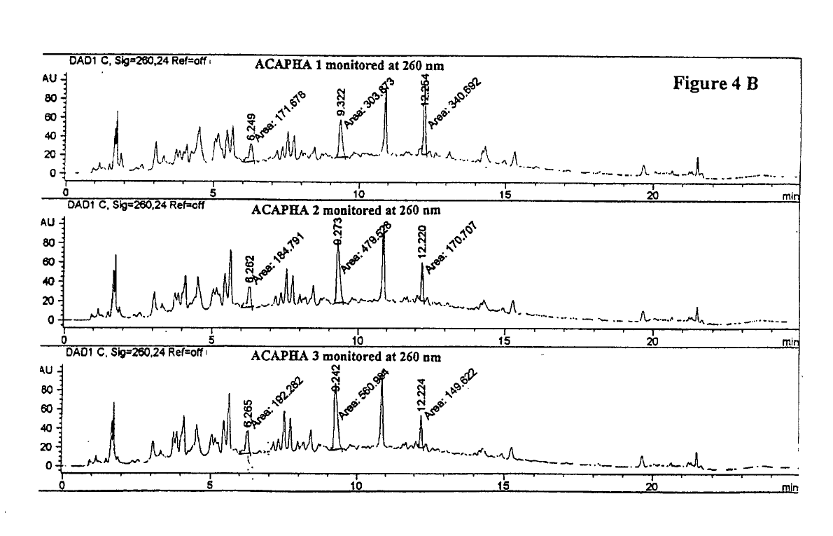

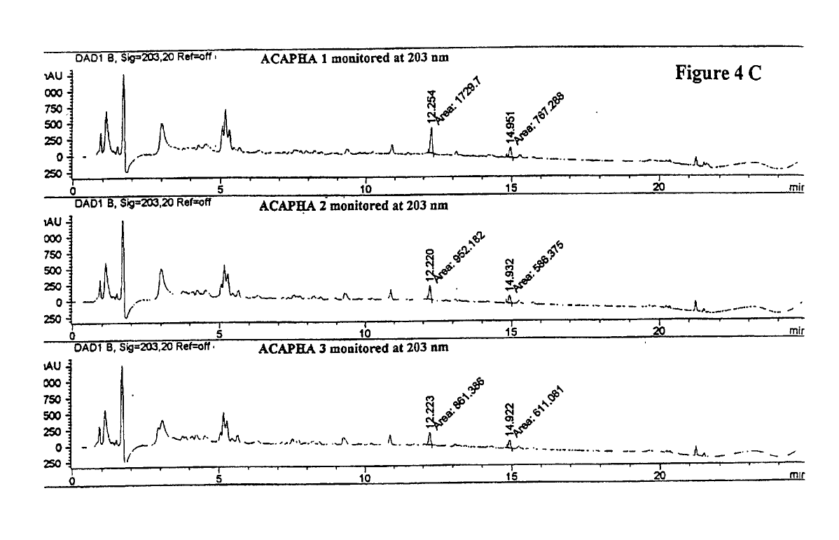

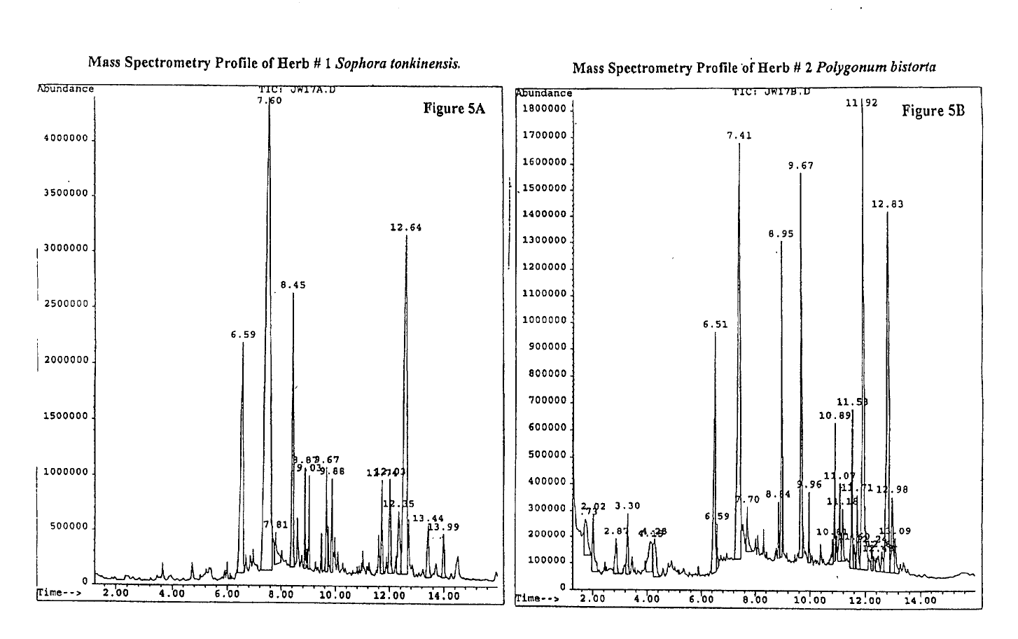

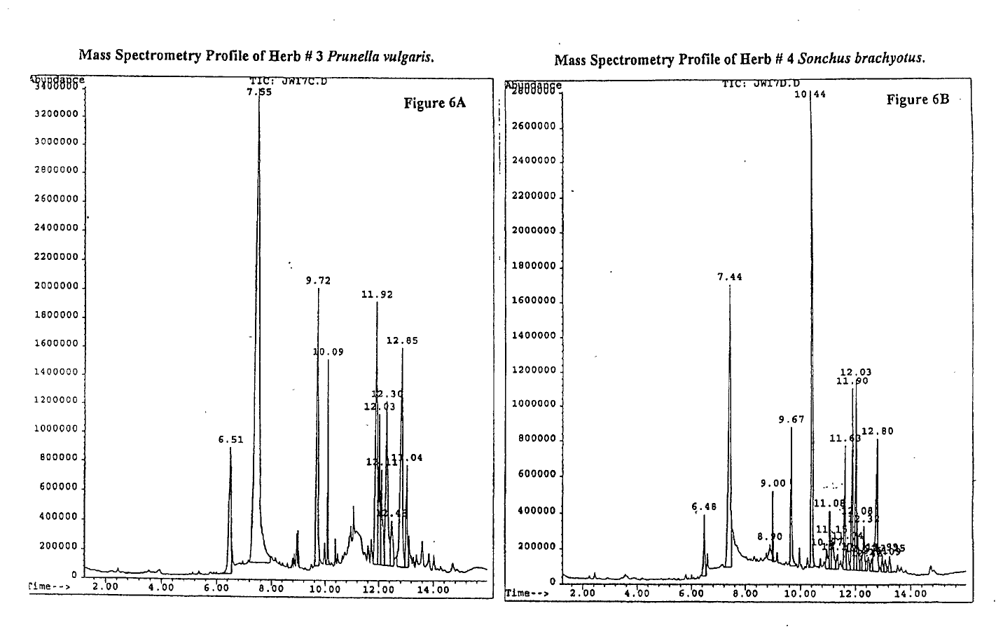

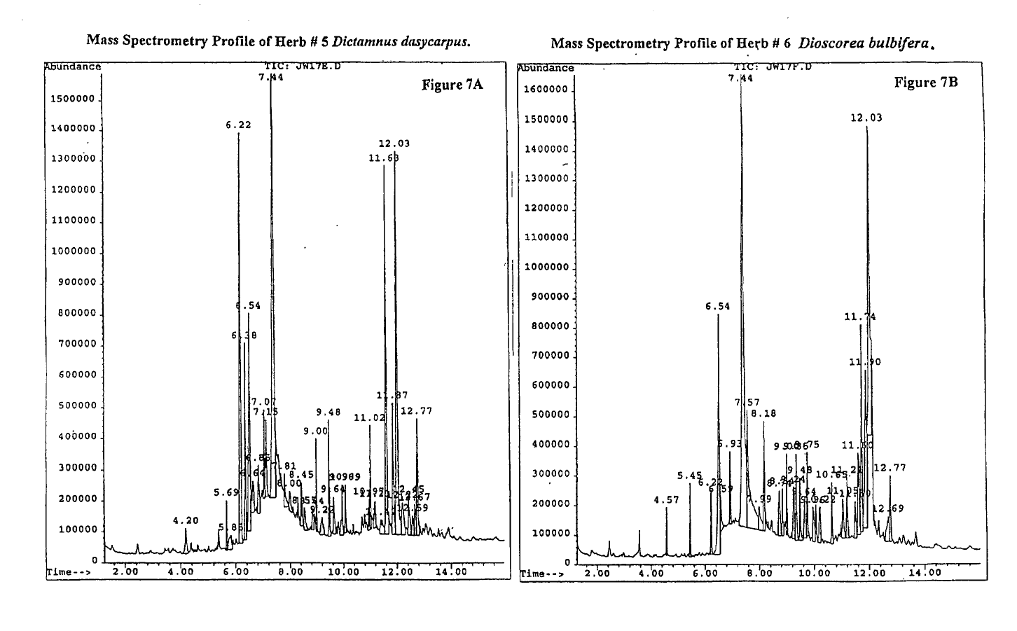

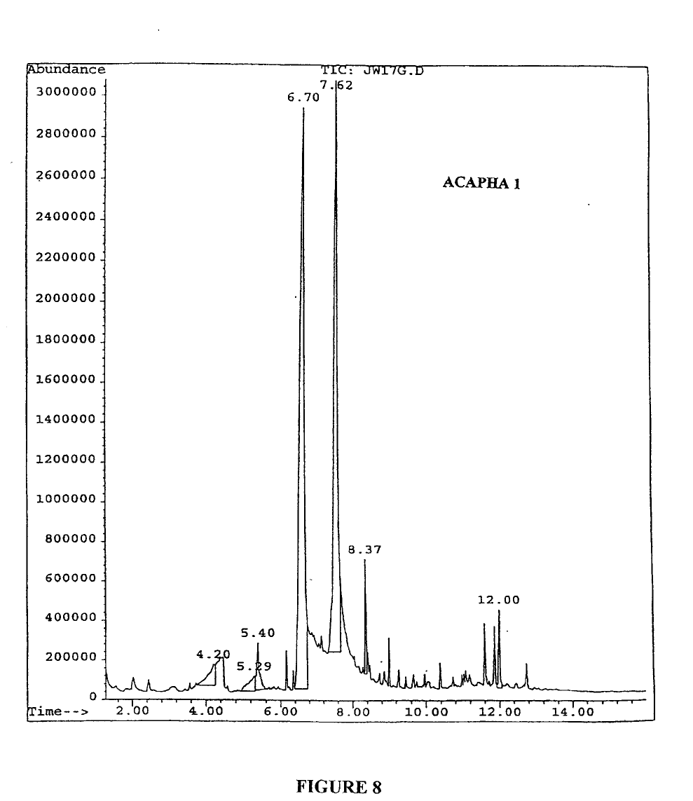

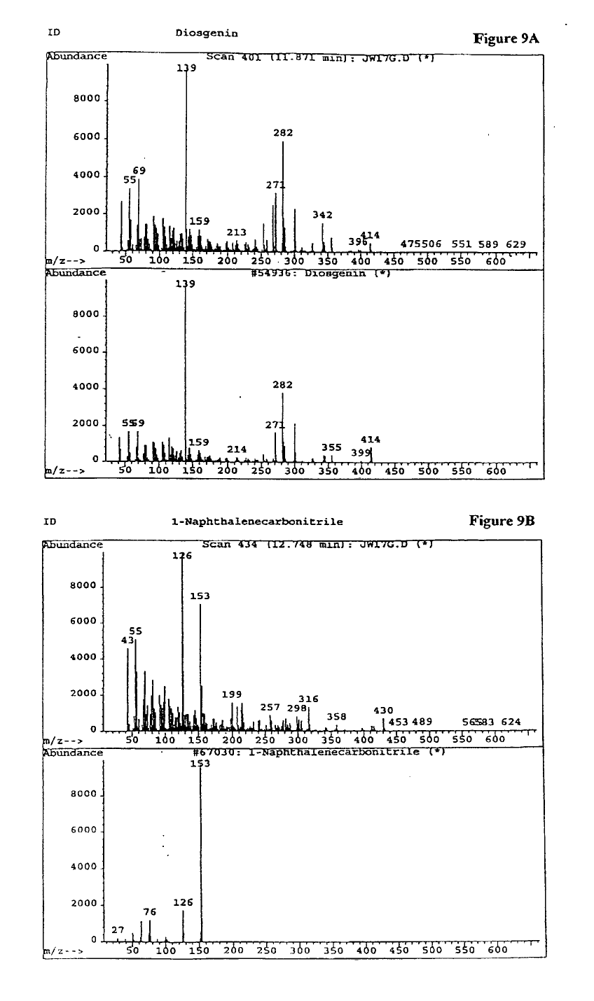

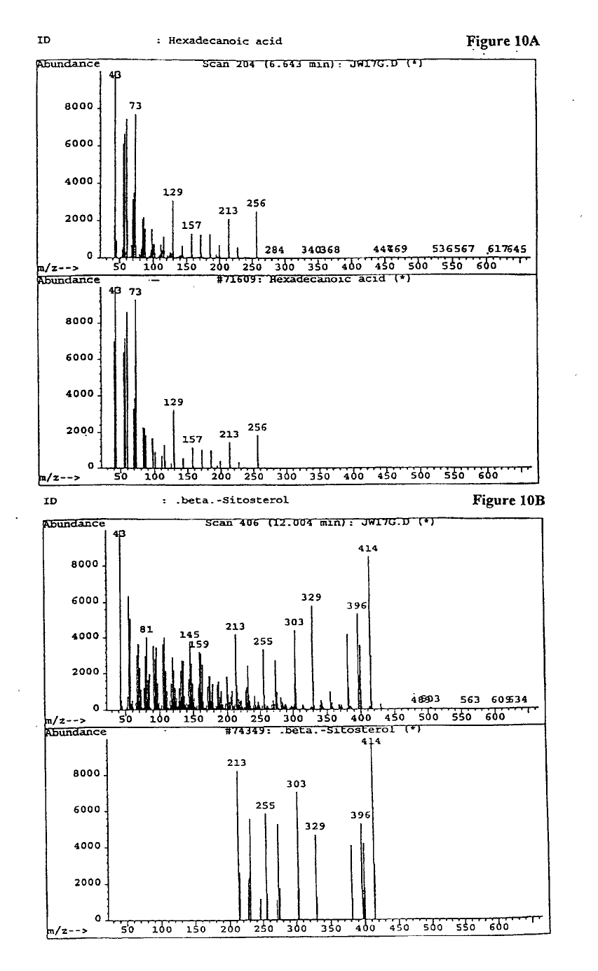

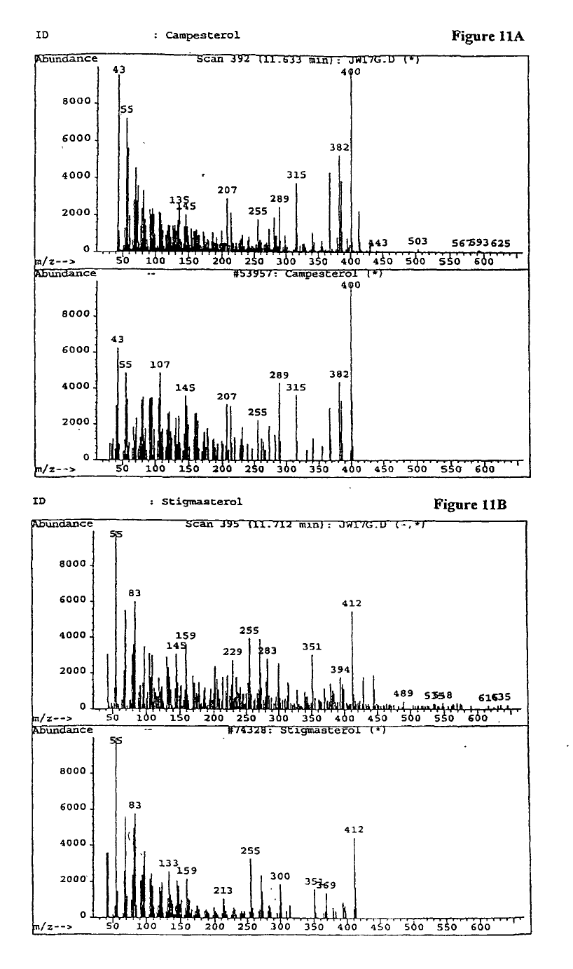

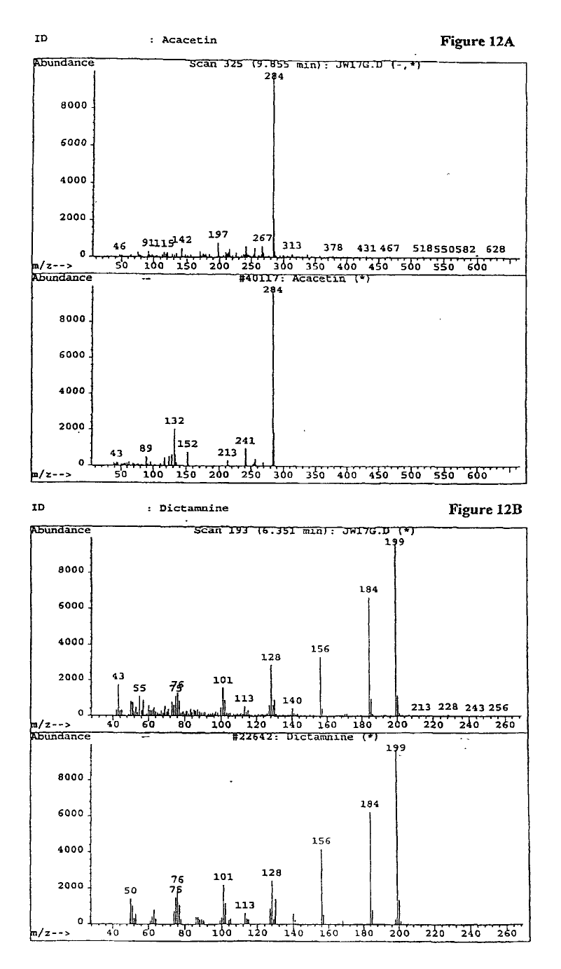

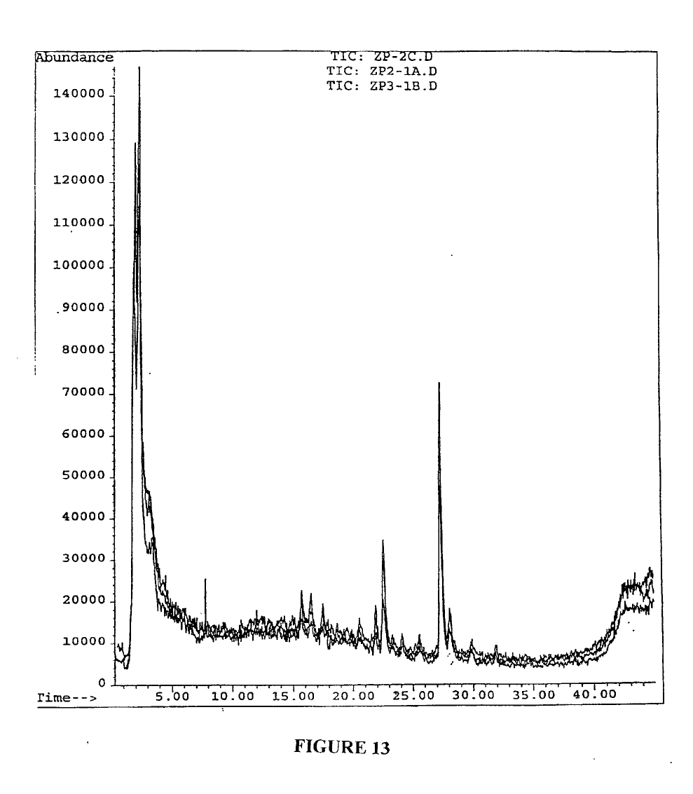

FIG. 1 shows the high performance liquid chromatography (HPLC) profiles of extracts of each of the six herbs, of ACAPHA. FIG. 2 illustrates the HPLC profile of all of the extracts of FIG. 1 overlaid. FIG. 3 illustrates the HPLC profile of the two different formulations of ACAPHA referred to above. FIG. 4 illustrates the HPLC profile of the three different formulations of ACAPHA referred to above. Fingerprint profiles and mass spectrometry studies (FIGS. 5-13) conducted to date suggest that ACAPHA comprises various organic acid compounds.

A number of safety tests and toxicology tests have been performed on ACAPHA. Chemical analyses have shown the heavy metals content to be within safety limits and microbiological studies were negative, ACAPHA was also found to be non-mutagenic with the Ames test. In chronic toxicity tests, no pathological changes were found in the heart, liver, spleen, lung, kidneys, adrenal glands, stomach, small intestine, pancreas, brain, lymph nodes, testicles and ovaries of rats and dogs despite high doses of ACAPHA. Animals treated with ACAPHA also showed no abnormal physical changes or effects on mobility, feeding, development and growth. There were also no obvious abnormal changes in the blood picture, urination, liver and kidney functions and ECG. Acute toxicity was reported to be very low as evidenced by a compliance rate of over 90% in a five-year clinical trial in over 1,000 subjects in China (described below). However, some minor side effects including an occasional increase in bowel movements, nausea, vomiting and skin rash have been observed. In some studies, ACAPHA treatment also resulted in the elevation of aspartate aminotransferase (AST) levels and lactic dehydrogenase (LD) levels in some human subjects. These effects usually subsided upon withdrawal of the medication. Also as shown below the elevation of these liver enzymes can be eliminated by use of the formulation ACAPHA 2, ACAPHA 3 or an equivalent formulation, wherein the Herb F is reduced in concentration or eliminated.

As previously noted, in one embodiment of the present invention, the tablets are formulated into 0.3g tablets. A typical daily dosage is about 1.2 to 6 g/day based on the body weight and/or severity of the condition.

Any suitable mode of delivery can be used according to the present invention. For example the composition of the present invention can be delivered orally by a pill, tablet, drink, candy or paste. The composition can also be delivered as a transdermal patch, by inhalation or suppository. Delivery of the composition as an injectable is also possible, according to the present invention. Therefore, the composition can be administered as a therapeutic agent or as a dietary supplement.

According to the present invention, the compositions are useful for the prevention and treatment of cancers. In one embodiment of the present invention; the cancers which are found to be treated or prevented are selected from the group consisting of lung cancer, esophagus cancer, stomach cancer, cancer of the oral cavity and prostate cancer. In a further embodiment of the present invention, the compositions have been found to be useful for the treatment of Helicobacter pylori infection. In yet a further embodiment of the present invention, the compositions have been found to be useful as adjuncts to conventional surgery or radiotherapy treatments in patients with cancer selected from but not limited to lung cancer, esophagus cancer, stomach cancer, oral cavity cancer and prostate cancer.

The present invention will now be illustrated by the following examples, which should not be considered limiting.

### EXAMPLES

### Example 1

### Effect of ACAPHA 1 on Esophageal Dysplasia

In 1983 an esophageal cytological examination was undertaken among 6,758 residents 40-65 years of age in Heshun Township, Lixian County, China; and among 2,875 residents in Leikou Township, Anyangxian County, China. A total of 2,531 cases with severe dysplasia were found in these townships.

Random grouping and inhibitory therapy of patients with severe esophageal dysplasia was undertaken. The treatment group was administered 8 tablets per day while the control group was treated with placebo. Esophageal cytological reexamination was conducted after three years of administration of ACAPHA 1 The reexamination rate amounted to 96.3%. The reexamination results are summarized in Table 1 below.

TABLE 1

| | | No. of subjects | |
|---|---|---|---|
| | | developing epithelial | |
| Group | No. of cases | cancer (3 years) | Percentage (%) |
| ACAPHA I | 744 | 24 | 3.2 |
| Control | 777 | 52 | 6.7 |

The results showed that the incidence rate of esophageal carcinoma among the 1521 total patients having severe dysplasia were 52.2% less in the ACAPHA 1 group as compared to the control group. This represents a very significant statistical difference (χ²=8.9115, p<0.01). Further, as shown in Table 2 below there was a clear reduction in the incidence rate in different age groups of severe dysplastic patients after ACAPHA 1 inhibitory therapy for three years.

TABLE 2

| | No. of | Cancerization | | | Cancerization | |
|---|---|---|---|---|---|---|
| | ACAPHA 1 | (ACAPHA 1 | Percentage | No. of Control | (Control | Percentage |
| Age Group | subjects | group) | (%) | subjects | group) | (%) |
| ˜40 | 106 | 1 | 0.9 | 95 | 0 | 0 |
| 40˜49 | 236 | 5 | 2.1 | 250 | 10 | 4 |
| 50˜59 | 296 | 13 | 4.4 | 318 | 25 | 7.9 |
| 60˜ | 106 | 5 | 4.7 | 114 | 17 | 14.9 |

An esophageal reexamination was repeated in 1989 after administration of ACAPHA 1 for five years. The reexamination rate was 93.7%. The results are summarized in Table 3 below.

TABLE 3

| | | No. of subjects | |
|---|---|---|---|
| | | developing epithelial | |
| Group | No. of cases | cancer (5 years) | Percentage (%) |
| ACAPHA 1 | 479 | 37 | 7.7 |
| Control | 507 | 74 | 14.6 |

The results showed that the incidence rate of esophageal carcinoma among the 986 total patients having severe dysplasia was 47.3% of the incidence rate of the control group. This represents a very significant statistical difference (χ²=10.9573, p<0.01).

Further, as shown in Table 4 below there was a clear reduction in the incidence rate in different age groups of severe dysplastic patients after ACAPHA 1 inhibitory therapy for five years.

TABLE 4

| | | Cancerization | | | | |
|---|---|---|---|---|---|---|
| | No. of ACAPHA | (ACAPHA 1 | Percentage | No. of Control | Cancerization | Percentage |
| Age Group | 1 subjects | group) | (%) | subjects | (Control group) | (%) |
| ˜40 | 31 | 1 | 3.2 | 24 | 1 | 4.2 |
| 40˜49 | 169 | 8 | 4.7 | 178 | 15 | 8.4 |
| 50˜59 | 199 | 19 | 9.5 | 220 | 37 | 16.8 |
| 60˜ | 80 | 9 | 11.3 | 85 | 21 | 24.7 |

In order to assess the forward effect of ACAPHA 1 inhibitory therapy, further observations were made 9 years after initiation of treatment. In particular, in the patient group initially having severe dysplasia administration of ACAPHA 1 was terminated after five years of treatment and these patients were thereafter administered riboflavin (5 mg/day) for four years. Patients in the control group remained taking placebo. An esophageal cytological reexamination was undertaken after 9 years of inhibitory therapy (treating for 5 years, stopping for 4 years). The reexamination rate was 89.3%. The results are summarized in Tables 5 and 6 below.

TABLE 5

| | | No. of subjects developing | |
|---|---|---|---|
| | | epithelial cancer (9 years- | |
| | | i.e. treating for 5 years, | |
| Group | No. of cases | stopping for 4 years) | Percentage (%) |
| ACAPHA 1 | 591 | 61 | 10.3 |
| Control | 630 | 112 | 17.8 |

TABLE 6

| | No. of ACAPHA 1 | Cancerization | Percentage | No. of Control | Cancerization | Percentage |
|---|---|---|---|---|---|---|
| Age Group | subjects | (ACAPHA 1 group) | (%) | subjects | (Control group) | (%) |
| ˜40 | 84 | 1 | 1.2 | 83 | 1 | 1.2 |
| 40˜49 | 201 | 168 | 8 | 206 | 27 | 13.1 |
| 50˜59 | 241 | 26 | 10.8 | 262 | 54 | 20.6 |
| 60˜ | 65 | 18 | 27.7 | 79 | 30 | 38 |

As indicated above, the results of esophageal cytological reexamination after 3-5 years of treatment indicated that the cancer ration rate of severe esophageal dysplasia in patients treated with ACAPHA 1 was reduced by approximately 50%. From the results of inhibitory therapy for nine years (treating for five years, stopping for four years), it can be seen the reduction rate in the ACAPHA 1 group remained approximately 40% as compared to the control group (χ²=13.94, p<0.01). It therefore appears that ACAPHA 1 not only has a marked effect on esophageal cancer prevention after long-term use, but also has a forward inhibitory effect even after cessation of therapy for as long as four years.

### Example 2

### Effect of ACAPHA 2 on Esophageal Dysplasia—Phase III Multicenter Trial

The incidence of esophageal cancer is closely related with esophageal epithelial dysplasia. Research into this type of dysplasia has resulted in the finding that esophageal epithelial dysplasia is an essential phase of esophageal epithelial carcinogenesis. Severe dysplasia is described as precancerous lesions of the esophageal epithelium. Severe dysplasia is a very unstable phase characterized by bi-directional transformation in which lesions can either progress to cancer or regress back to normal. Patients with severe dysplasia constitute a group of individuals at high-risk of developing esophageal cancer. In this example, ACAPHA 2 was used in a single-blind, randomized, multicenter phase III clinical trial in China to examine its effects on patients with esophageal dysplasia over a six-month period.

Patients were recruited from Hebei province, China, and were between the ages of 40 and 65 years old. Following an initial interview, an esophageal cytological assessment was performed. Those patients with severe esophageal dysplasia were further investigated with esophageal endoscopy and biopsy for histopathology to confirm the diagnosis. Biopsies were taken from areas with abnormal appearances. Patients with obvious heart, liver or lung disorders, liver cirrhosis, venous hypertension, and esophageal and inferior gastric venous distention were excluded from the trial.

A total of 514 patients were recruited for the study. Of those, 449 cases, 300 in the treatment group and 149 in the control group, were available for reassessment and evaluation of the treatment effect. The patients were randomized and stratified according to gender, age, and severity of esophageal dysplasia (mild, moderate or severe).

The treatment and control groups took sugar-coated tablets of the same appearance. Each bottle contained 80 tablets of 0.3 g each. The patients were instructed to take 8 tablets of ACAPHA 2 or placebo, twice a day for 6 months. Study personnel visited each patient at home on a monthly basis to dispense the medication. During the visit, the number of pills left unused from the previous month was counted. At the end of the treatment period, the actual quantity of unused drug was compared to the total amount of drug that the subjects should have taken for the duration of the trial. Any side effects or adverse reactions experienced by the patients were recorded. To objectively evaluate patients' compliance in taking the medication, urinary riboflavin testing was employed. On the third month of the treatment, all patients in the ACAPHA 2 treatment group were supplied with ACAPHA 2 containing riboflavin (5 mg/8 tablets) for 15 days. Thereafter supply of regular ACAPHA 2 resumed. On the last day of the riboflavin-ACAPHA 2 (day 15), afternoon urine sample was collected from all the patients for determination of urinary riboflavin concentration. According to Chinese laboratory reference, the 4 hour urinary riboflavin content is as follows: <400 μg is deficient; 400-799 μg is inadequate; 800-1299 μg is normal and >1,300 μg is abundant.

During the course of the trial period, patients were given monthly physical examinations and clinical symptoms associated with the disease were recorded. In addition, blood picture, liver function tests, serum glutamic pyruvate transaminase (SGPT, bilirubin, bleeding and clotting times), kidney function test, blood urea nitrogen (BUN), and ECG were carried out before, 3 and 6 months after treatment was started. Finally, esophageal endoscopy and biopsy for histopathology to objectively compare the histological changes were carried out before and after treatment.

The evaluation criteria for esophageal epithelial dysplasia were as follows. Endoscopic appearances were classified into 4 grades: inflammation, mild, moderate and severe dysplasia. Inflammation was defined as mucosal necrosis with whitish coloration of mucosa and a well-vascularized reflective surface. Dysplasia was defined as a localized esophageal mucosal epithelial surface with rough and greyish white specks, and lumps/lumpy projections above the mucosal surface. Based on the distribution and size of involvement and the thickness of the mucosa, the degree of dysplasia was further classified into mild, moderate and severe grades.

Histopathological assessment criteria of biopsy was classified into 7 grades: inflammation, hyperplasia (mild, moderate and severe) and a typical dysplasia (mild, moderate and severe). Inflammation was defined as mucosal and submucosal layers have a varying extent of inflammatory cell infiltration and distension of blood vessel in the submucosal layer.

Hyperplasia was defined as an increase in the number of superficial layers several times that of normal tissue. This was mainly due to the hyperplasia or hypertrophy of basal cells. Cells in the lower layers showed spindle shape. The longitudinal section showed an increase in nuclear size, hyperchromatism of nuclei, and irregular shapes of cells with disorderly arrangement. According to the increase in the number of upper layers in comparison with the normal condition, the hyperplastic changes were classified into mild, moderate and severe categories. Increase in the upper cell layer by 2 fold is mild hyperplasia; increase by 5 fold is severe hyperplasia and an increase somewhere in-between is moderate hyperplasia.

Atypical dysplasia is defined as cells in the upper layer showing abnormal mitotic figures, disorderly arrangement of the cells within the epithelium, variation in the size and shape of the cells, enlargement, irregularity, and hyperchromatism of the nuclei. The increase in the number of mitotic figures was used to classify the dysplasia. Cases that involved less than ⅓ of the epithelial layer was graded as mild dysplasia, over ⅓ involvement was graded as moderate dysplasia and over ½ involvement was graded as severe dysplasia.

After the completion of the clinical trial, all patients received reassessment by esophageal endoscopy and biopsy for histopathology. The changes were categorized into improvement, stable and progressive disease. Improvement was defined as reduction of the disease condition by 1 or more grades, stable was defined as no change in the disease condition, and progressive disease was defined as an increase in the disease condition by 1 or more grades.

Ridit-Analysis was applied to a contingency table analysis to determine the treatment effect with ACAPHA 2 or placebo. Pearson's Chi-square test was applied to compare the difference in the patient population in terms of gender, age, severity of esophageal changes in the treatment and placebo group. A p value of less than 0.05 was considered statistically significant.

A total of 494 participants fulfilled the inclusion/exclusion criteria and were admitted into the study. A total of 449 cases were available for evaluation with an overall reassessment rate of 90.9%. The reassessment rates for the placebo control and ACAPHA 2 treatment groups were 88.7 and 92% respectively. Reasons for not completing the full length of the trial include refusal to take the medication, death due to causes other than cancer, failure to attend scheduled clinic visits, away during scheduled home visits and cannot continue to participate in the study due to serious illness. However, none of these can be attributed to the treatment protocol or due to side effects.

Patient demographics are shown in Tables 7 and 8. Table 7 and FIG. 14A and FIG. 14B) show that there was no difference in the distribution of age and gender between the ACAPHA 2 and placebo treatment groups. Table 8 shows that the distribution of the patients with different grades of pathological changes as assessed by esophageal biopsy histopathology was similar between the ACAPHA and placebo treatment groups.

TABLE 7
Patient distribution according to age and gender*

| | | Placebo Control | | ACAPHA 2 Treatment | |
|---|---|---|---|---|---|
| | | (n = 149) | | (n = 300) | |
| | | Number | Percent | Number | Percent |
| Age | 40-49 | 60 | 40.3 | 111 | 37 |
| | 50-59 | 56 | 37.6 | 115 | 38.3 |
| | >60 | 33 | 22.1 | 74 | 24.7 |
| Gender | Male | 69 | 46.3 | 139 | 46.3 |
| | Female | 80 | 53.7 | 161 | 53.7 |

*Placebo control group vs ACAPHA 2 treatment group, χ² for age and gender are 0.56 (V = 2) and 0.00002 (V = 1) respectively, p > 0.05, non significant.

TABLE 8
Patient distribution in staging of disease according to assessment by
biopsy histopathology*

| | | Placebo Control | | ACAPHA 2 Treatment | |
|---|---|---|---|---|---|
| Assessment | | (n = 149) | | (n = 300) | |
| Method | Staging | Number | Percent | Number | Percent |
| Biopsy | Mild | 54 | 36.2 | 80 | 26.7 |
| histopathology | hyperplasia | | | | |
| | Moderate | 23 | 15.4 | 44 | 14.7 |
| | hyperplasia | | | | |
| | Severe | 7 | 4.7 | 9 | 3.0 |
| | hyperplasia | | | | |
| | Mild | 36 | 24.2 | 93 | 31.0 |
| | dysplasia | | | | |
| | Moderate | 23 | 15.5 | 57 | 19.0 |
| | dysplasia | | | | |
| | Severe | 6 | 4.0 | 17 | 5.6 |
| | dysplasia | | | | |

*Patient staging in placebo control vs ACAPHA 2 treatment group, χ² for assessment based on biopsy histopathology is 0.07 (V = 2), p > 0.05, non significant.

In addition, excellent participant compliance rate for taking ACAPHA 2 was observed. Table 9 shows that 99% of patients took the medication according to treatment schedule as determined by pill count at monthly intervals. To ensure the validity of the pill count and eventually the outcome data, an indicator agent (riboflavin) was included in the tablets taken at regular intervals. Of the 300 patients in the ACAPHA 2 treatment group, 297 (99%) have urinary riboflavin content of >1000 μg. According to the nutritional assessment of the residents of the Cixion county, the vast majority of the residents are severely deficient in riboflavin. The urinary riboflavin measurements of the ACAPHA 2 treated participants suggest excellent compliance and corroborating the pill counts.

TABLE 9
Statistics of left over medications for the control and treatment groups.

| | Patient with left over med. | | | Quantity of left over med. | | |
|---|---|---|---|---|---|---|
| | | | | Total | | |
| | # of | Total | | tablets | Left over | |
| Group | patients | case | % | (x10,000) | tablets | % |
| Control | 6 | 894 | 0.7 | 42.9 | 343 | 0.08 |
| Treatment | 8 | 1800 | 0.4 | 86.4 | 1062 | 0.12 |

The main clinical symptoms of patients with esophageal dysplasia include: dry mouth, dry throat, gastric reflux, burning sensation of the stomach, bloating of the stomach, stomach discomfort, dry feces, with some patients feel discomfort in swallowing.

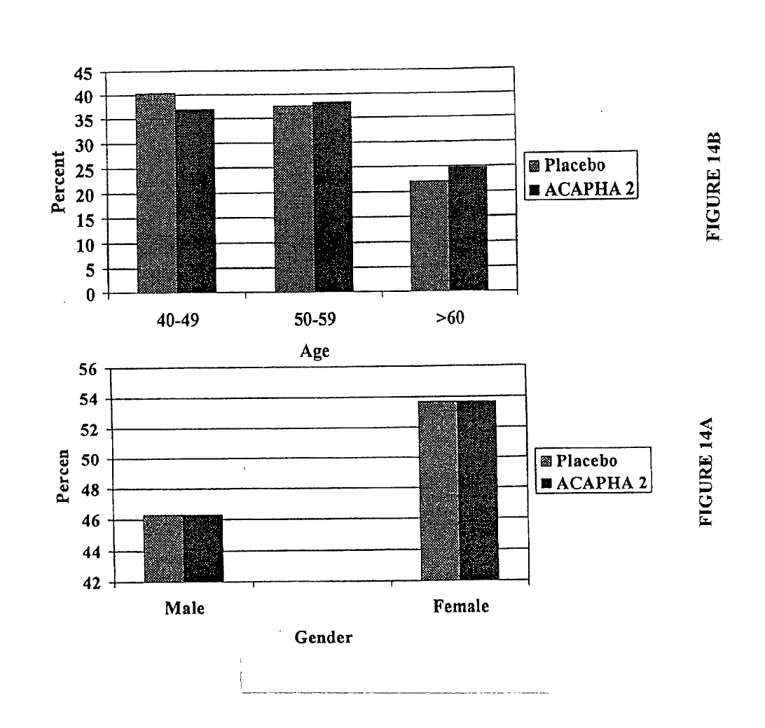

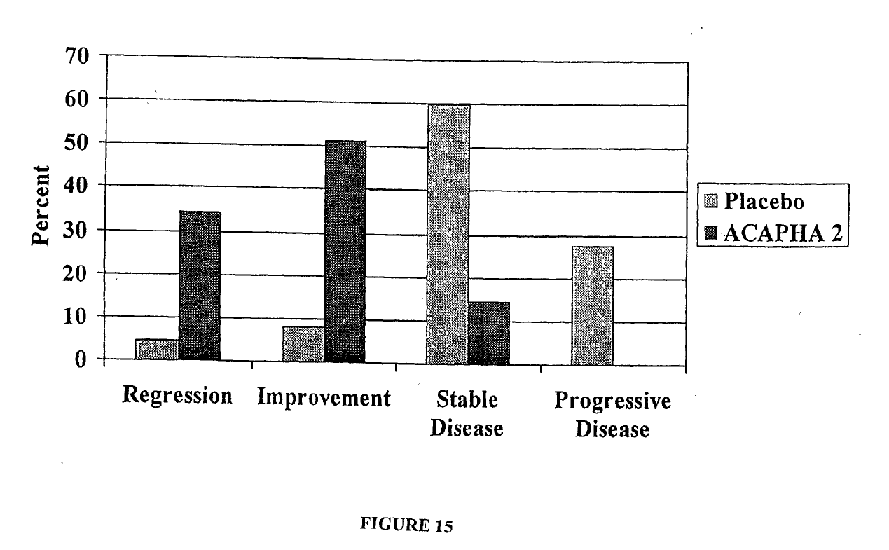

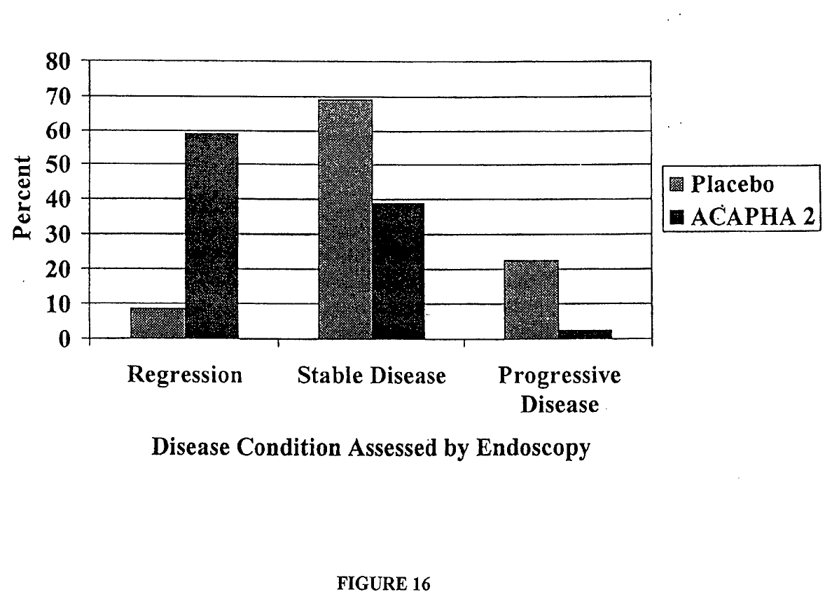

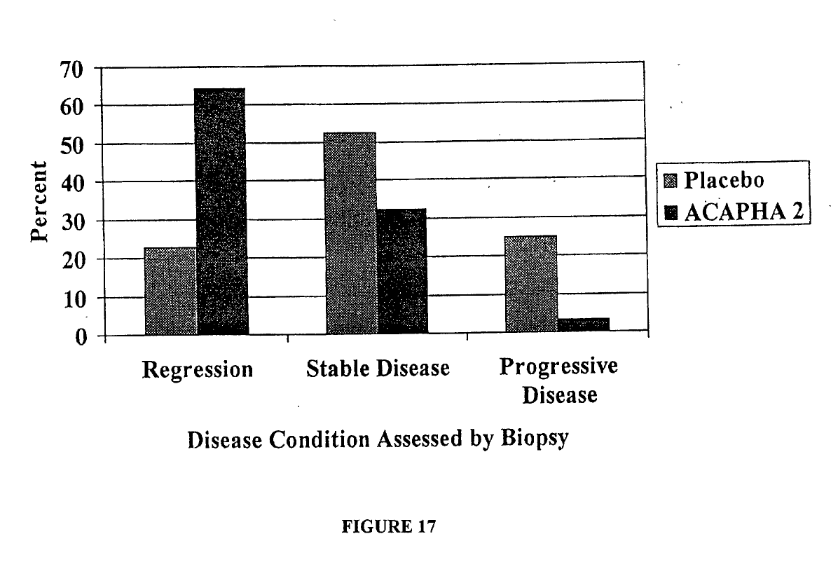

The therapeutic effects are shown in FIGS. 15, 16 and 17 and Table 10. The ACAPHA 2 treated group had significant improvements by all three assessments compared with the control group.

TABLE 10
Analysis of treatment effect of ACAPHA 2 according to clinical
symptoms, endoscopy and biopsy histopathology.

| | | Control Group | | Treatment Group | | |
|---|---|---|---|---|---|---|
| | | (n = 149) | | (n = 300)* | | Ridit value |
| Assessment Method | Condition | Number | Percent | Number | Percent | (95% C.I) |
| Clinical symptoms | Regression | 7 | 4.7 | 103 | 34.4 | 0.11 |
| | Improvement | 12 | 8.1 | 154 | 51.3 | (0.10˜0.15) |
| | Stable | 89 | 59.7 | 43 | 14.4 | |
| | Progressive Disease | 41 | 27.5 | O | 0 | |
| Endoscopy | Improvement | 13 | 8.7 | 177 | 59 | 0.21 |
| | Stable | 103 | 69.1 | 116 | 38.7 | (0.18˜0.25) |
| | Progressive | 33 | 22.2 | 7 | 2.3 | |
| | disease | | | | | |
| Biopsy histopathology | Improvement | 34 | 22.8 | 193 | 64.4 | 0.26 |
| | Stable disease | 78 | 52.4 | 97 | 32.3 | (0.23˜0.29) |
| | Progressive disease | 37 | 24.8 | 10 | 3.3 | |

*By clinical symptoms, esophageal endoscopy and biopsy histopathology, results in the ACAPHA 2 treatment group were significant different from the placebo control group by Ridit analysis.

By clinical symptoms measured at the end of the 6 month trial, regression was observed in 34.4% (n=103) of ACAPHA 2 treated participants versus 4.7% (n=7) of those in the placebo control group. ACAPHA 2 treatment also resulted in >85% of patients having improvement of clinical symptoms, significantly higher than the 12.8% observed in the placebo control group. By clinical symptoms, esophageal endoscopy and biopsy histopathology, improvement rates were at 51.3%, 59% and 64.4% respectively in the ACAPHA 2 treated participants, significantly higher than 8.1%, 8.7% and 22.8% respectively in those on placebo control group by Ridit analysis. Disease progression rates measured by clinical symptoms, endoscopy and histopathology were 0%, 2.3% and 3.3% in the ACAPHA 2 treated participants, significantly lower than 27.5%. 22.2% and 24.8% respectively in the placebo control group by Ridit analysis.

The efficacy of treatment based on the pretreatment endoscopy condition of the patients was further analyzed and the results are present in Table 11. Table 11 shows the endoscopy results of patients in the various disease staging by endoscopy were evaluated by RIDIT statistical analysis using placebo groups as reference. There was statistically significant difference in disease staging by endoscopy following treatment between the ACAPHA 2 and placebo treatment groups.

TABLE 11
Endoscopic findings after treatment with ACAPHA 2 or placebo.

| | | | | | | | Progressive | | |
|---|---|---|---|---|---|---|---|---|---|
| | | | Improvement | | No change | | Disease | | Ridit Value* |
| GROUP | | Number | # | % | # | % | # | % | 95% C.I. |
| Mild Dysplasia | Placebo | 15 | 1 | 6.7 | 5 | 33.3 | 9 | 60.0 | 0.30 |
| | ACAPHA 2 | 16 | 10 | 62.5 | 6 | 37.5 | 0 | 0 | (0.15-0.44) |
| Moderate Dysplasia | Placebo | 38 | 3 | 7.9 | 18 | 47.4 | 17 | 44.7 | 0.19 |
| | ACAPHA 2 | 81 | 48 | 59.3 | 26 | 32.1 | 7 | 8.6 | (0.13-0.26) |
| Severe Dysplasia | Placebo | 96 | 9 | 9.4 | 80 | 83.3 | 7 | 7.3 | 0.24 |
| | ACAPHA 2 | 203 | 119 | 58.6 | 84 | 41.1 | 0 | 0 | (0.20-0.28) |

*Based on Ridit analysis, there was statistically significantly difference following treatment between the ACAPHA 2 and placebo treatment groups.

Table 12 shows the relationship between biopsy histopathology assessment results of ACAPHA 2 treatment group with different initial disease staging. Based on the Ridit analysis using placebo group as reference, there was stastically significant difference between the placebo and ACAPHA 2 treatment groups.

TABLE 12
Analysis of treatment effect of ACAPHA2 according to biopsy histopathology*.

| | | | | | Stable | | Progressive | | |
|---|---|---|---|---|---|---|---|---|---|
| | | | Improvement | | Disease | | Disease | | Ridit Value |
| GROUP | | Number | # | % | # | % | # | % | 95% C.I. |
| Mild | Placebo | 36 | 8 | 22.2 | 23 | 63.9 | 5 | 13.9 | 0.27 |
| Dysplasia | ACAPHA 2 | 93 | 62 | 66.7 | 27 | 29.0 | 4 | 4.3 | (0.21-0.33) |
| Moderate | Placebo | 23 | 11 | 47.8 | 7 | 30.5 | 5 | 21.7 | 0.37 |
| Dysplasia | ACAPHA 2 | 57 | 38 | 66.7 | 18 | 31.6 | 1 | 1.7 | (0.30-0.45) |
| Severe | Placebo | 6 | 3 | 50.0 | 3 | 50.0 | 0 | 0 | 0.31 |
| Dysplasia | ACAPHA 2 | 17 | 15 | 88.2 | 2 | 11.8 | 0 | 0 | (0.17-0.45) |

*Based on Ridit analysis, there was statistically significant difference following treatment between the ACAPHA 2 and placebo treatment groups.

All the participants tolerated ACAPHA 2 well. Liver enzyme SGPT and bilirubin concentrations were within normal range in all the samples tested before, three and six months after ACAPHA 2 treatment (a=300 participants). Borderline elevation of BUN (normal range 3.2-7.0 mmol/L; borderline elevation range 7.1-9.9 mmol/L) was detected in 3 and 12 participants at three and six months after ACAPHA 2 treatment respectively. None of the participants who had borderline elevated BUN at 3 months also had borderline elevated level at 6 months. There were no abnormal changes in blood picture, ECG at 3 and 6 months after treatment, in comparison with pretrial data. Several patients have increased number of defecation, nausea, eczema but the condition disappeared once the medication was stopped.

In this Phase III (Chinese) multicenter study, ACAPHA 2 showed a very good effect in treating precancerous lesions and inflammation of esophagus. The progression of esophageal dysplasia was prevented effectively by ACAPHA 2. Relief of clinical symptoms was significantly higher in the ACAPHA 2 treated group than the placebo controls. The canceration of dysplasia of esophageal epithelium was obviously inhibited by ACAPHA 2. The compliance rate was 99% in the ACAPHA 2 treatment group as indicated by both the pill counts and urinary riboflavin determination. These results suggested that ACAPHA 2 is an effective chemoprevention drug with low toxicity.

### Example 3

### Use of ACAPHA 1 as an Adjunctive Therapy for Treatment of Esophageal Cancer

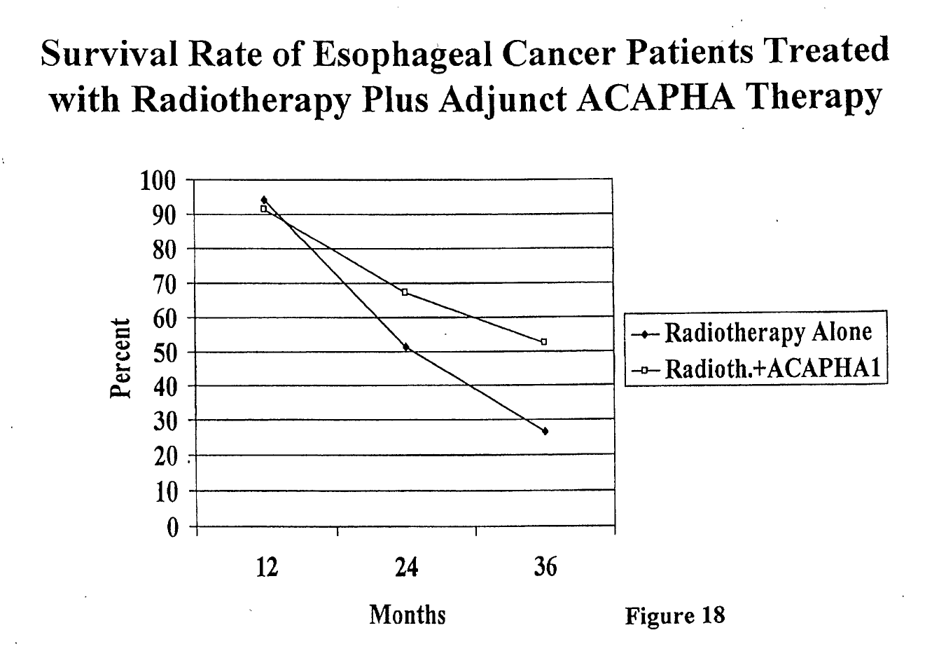

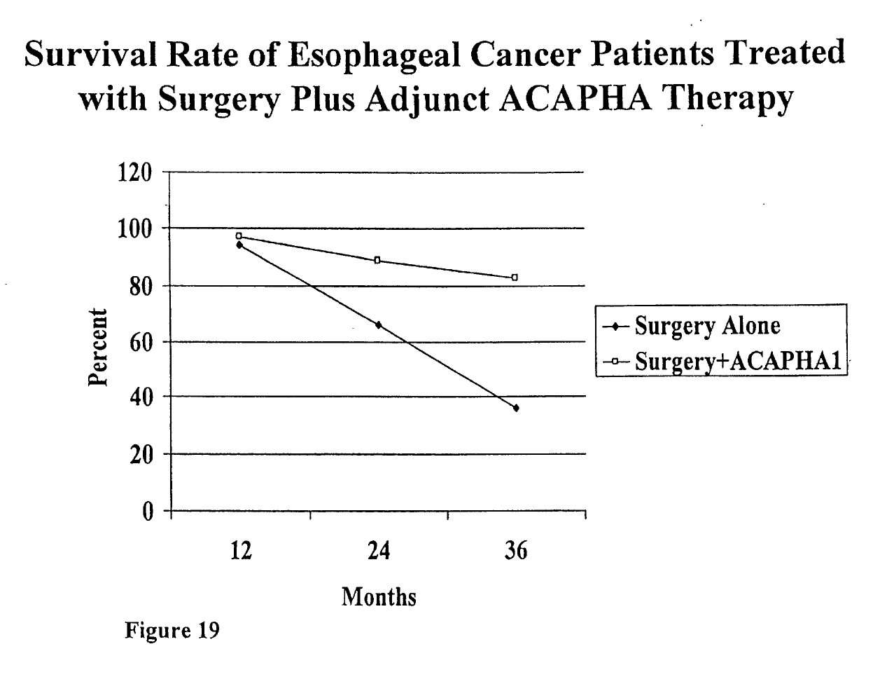

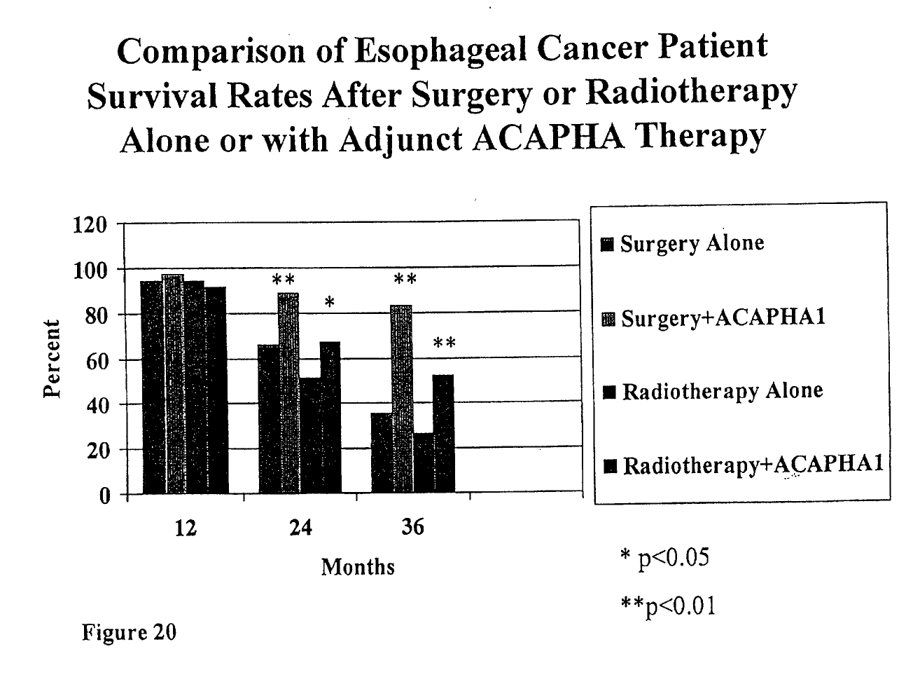

A five year trial in China commenced in January, 1997 to assess the efficacy of ACAPHA 1 as an adjunctive therapy for treatment of esophageal carcinoma. According to this trial, patients diagnosed with esophageal carcinoma were either treated by surgery or radiotherapy in conjunction with ACAPHA 1 administration. Historical data of patients treated for esophageal cancer by surgery or radiotherapy at the same cancer hospital was used for comparison. Survival rates in patient subjects twelve, twenty-four and thirty-six months after treatment commencement are summarized in FIGS. 18, 19 and 20 and in Tables 13 and 14. As shown in the Figures, the highest survival rate was in patients receiving ACAPHA 1 treatment in combination with surgery. Patients receiving ACAPHA 1 treatment in combination with radiotherapy also had a significantly higher survival rate as compared to patients receiving radiotherapy alone. The difference in survival rate between patients receiving conventional treatment and patients receiving ACAPHA 1 adjunctive therapy becomes more pronounced the longer the time period elapsed. For example, three years post-surgery, the survival rate of the 104 patients receiving surgery only was only 35.8%. By comparison, 82.9% of the 123 patients receiving a combination of surgery and ACAPHA 1 adjunctive therapy survived to the three year milestone.

TABLE 13
Summary of clinical data comparing esophageal cancer staging in patients treated with ACAPHA 1 as an
adjunct to conventional surgery or radiotherapy

| | | Stage I | | Stage II | | Stage III | | Stage IV | | |
|---|---|---|---|---|---|---|---|---|---|---|
| Group | N | N | % | N | % | N | % | N | % | Statistical Analysis |
| Surgery + ACAPHA 1 | 123 | 7 | 5.7 | 47 | 38.2 | 63 | 51.2 | 6 | 4.9 | χ² = 7.72 p > 0.05 |
| Surgery Alone | 104 | 3 | 2.9 | 51 | 49.0 | 50 | 48.1 | — | — | |
| Radiotherapy + ACAPHA 1 | 94 | 2 | 2.1 | 37 | 39.4 | 48 | 52.1 | 6 | 6.4 | χ² = 1.41 p > 0.05 |
| Radiotherapy Alone | 87 | 7 | 8.1 | 27 | 31.0 | 53 | 60.9 | — | — | |

TABLE 14
Comparison of survival rates of esophageal cancer patients treated with
ACAPHA 1 as an adjunct to conventional surgery and radiotherapy
36 months post-treatment

| | | Survival Rate (%) | | |
|---|---|---|---|---|
| Treatment | Months Post-Rx | Plus ACAPHA 1 | | U-Test |
| Surgery | 12 | 97.2 | 94.2 | U = 1.09 p > 0.05 |
| | 24 | 88.9 | 66.1 | U = 3.82 p < 0.01 |
| | 36 | 82.9 | 35.8 | U = 6.04 p < 0.01 |
| Radiotherapy | 12 | 91.3 | 94.2 | U = 0.74 p > 0.05 |
| | 24 | 67.2 | 51.3 | U = 2.56 p < 0.05 |
| | 36 | 52.3 | 26.4 | U = 2.71 p < 0.01 |

The 5 year trial started in January 1997. The data summarized in the table represent results ending June 2000. The survival statistics were analyzed with Mann-Whitney U-Test.

### Example 4

### Effect of ACAPHA 1 on Bronchial Dysplasia

Bronchial dysplasia is a premalignant lesion of the bronchial epithelium. Currently there is no standard treatment for bronchial dysplasia Because tobacco use is one of the major causes of lung cancer, and former heavy smokers retain an elevated risk for lung cancer even years after they have stopped smoking, the effects of ACAPHA 1 on bronchial dysplasia in former smokers was investigated.

Twenty current and former smokers over 40 years of age with a smoking history of at least 30 pack-years (i.e. 1 pack per day for 30 years or more) and one or more sites of bronchial dysplasia on fluorescence bronchoscopy directed bronchial biopsies were recruited to take part in the study. Participants from the placebo arm of two concurrent chemoprevention trials with identical inclusion and exclusion criteria and similar clinical protocols aside from the chemopreventive agent were used for comparison purposes.

Following an initial interview, which included a questionnaire to document the smoking history, fluorescence bronchoscopy using the LIFE-Lung device (Xillix Technologies Corp., Richmond, B.C, Canada) was performed on each patient. Biopsies were taken from areas with abnormal fluorescence. In addition, at least two control biopsies were taken from an upper or lower lobe. A minimum of four biopsies were taken per individual.

The participants were instructed to take ACAPHA 1 at a dose of 8 tablets, twice a day for 6 months. They were seen monthly for examination of drug related adverse effects. Complete blood counts, AST, LD, bilirubin, alkaline phosphatase, calcium, phosphate, electrolytes, BUN, creatinine, triglycerides and cholesterol were measured at baseline and monthly for 6 months while on ACAPHA 1 for toxicity monitoring. Fluorescence bronchoscopy was repeated after 6 months of ACAPHA 1 treatment. All previously biopsied sites were identified and re-biopsied under fluorescence bronchoscopy. Biopsies were also taken from new areas that appeared to be dysplastic under fluorescence examination.

The biopsies were fixed in buffered formalin, embedded in paraffin, sectioned and stained with hematoxylin and eosin All the biopsies were systematically reviewed by an experienced pathologist. All biopsies were classified into one of eight groups. The normal group was represented by pseudostratified ciliated columnar epithelium. The basal cell hyperplasia group was represented by an increase in the number and stratification of normal-appearing basal cells still covered with normal ciliated or mucin secreting cells. The metaplasia group was represented by a stratified epithelium and cytoplasmic changes consistent with squamoid differentiation. Mild, moderate or severe dysplasia and carcinoma in-situ were classified according to WHO criteria. The final group was classified as invasive, metastatic carcinoma.

The primary end-point of the study (i.e. surrogate end-point biomarker) was change in the histopathology grade based on the risk of progression to invasive cancer. From the study by Frost and co-workers (J. Occp. Med 1986; 28:692-703), the risk of progression of mild, moderate and severe dysplasia to invasive lung cancer within nine years were 4%, 10%, and 40%, respectively. On a site by site analysis, complete response (CR) was defined by regression of the dysplastic lesion to hyperplasia/normal. Progressive disease (PD) was defined as appearance of lesions that were mild dysplasia or worse irrespective of whether the site was biopsied at baseline or worsening of the dysplastic lesions present at baseline by two grades or more (i.e. mild dysplasia to severe dysplasia or worse, moderate/severe dysplasia to carcinoma in-situ/invasive cancer). No response (NR) referred to sites that were not a CR or PD.

On a participant by participant basis, CR was defined as regression of all dysplastic lesions found at baseline to no higher than hyperplasia as defined by the site by site analysis at six months and no appearance of new dysplastic lesions that were mild dysplasia or worse. PD was defined as progression of one or more sites to a higher grade as defined by the site by site analysis referred to above or appearance of new dysplastic lesions that were mild dysplasia or worse at six months. Partial response (PR) was defined as regression of some but not all of the dysplastic lesions but no appearance of new lesions that were mild dysplasia or worse. No response (NR) referred to subjects who did not have a CR, PR or PD.

Descriptive statistics were used to summarize subject characteristics and pathologic evaluations of the bronchial biopsy examinations. Comparison between groups was done with the Mann-Whitney test. Pearson's Chi-square test was applied to a contingency table analysis to determine the association of bronchial dysplasia with treatment (ACAPHA 1 or placebo). All P values are two-sided. A two-sided P value of less than 0.05 was considered to be statistically significant.

To adjust for the effect of various factors on the likelihood of regression of preinvasive lesions, a multiple logistic regression analysis was used. The analysis included the variables: age, sex, and the smoking intensity (pack-years). All analyses were unconditional and tests of statistical significance and confidence intervals (CIs) for odds ratios (ORs) were based on the log-likelihood test.

The characteristics of the participants are shown in Table 15. There was no significant differences in age, sex and smoking history between the ACAPHA 1 and placebo groups. There was also no significant difference in the number of biopsies taken, the proportion of dysplastic lesions or the severity of the dysplastic lesions between the two groups. A total of 421 biopsies were taken in the placebo group (average 5.8 biopsies per subject). Twenty-four of the 421 biopsies (5.7%) were moderate/severe dysplasia and 168 biopsies (39.9%) showed mild dysplasia In the ACAPHA 1 group, a total of 121 biopsies were taken (average 6.1 per subject). Ten of the 121 biopsies (8.3%) were moderate/severe dysplasia and 36 biopsies (29.8%) showed mild dysplasia.

TABLE 15
Characteristics of participants in the Phase IIA Trial of the Effect of
ACAPHA 1 on Bronchial Dysplasia

| | | ACAPHA 1 | Placebo* |
|---|---|---|---|
| Number of Participants | | 20 | 73 |
| Age (Years) | Median | 58 | 55 |
| | Range | 46-71 | 43-72 |
| Gender | Male | 12 | 44 |
| | Female | 8 | 29 |
| Smoking | Former | 6 | 11 |
| Status | Current | 14 | 62 |
| Pack-Years | Mean | 65 | 46 |
| | Range | 30-85 | 30-72 |

*Placebo data are drawn from two concurrent Phase IIb placebo-controlled trials evaluating an identical cohort treated under the identical protocol at the same institution.

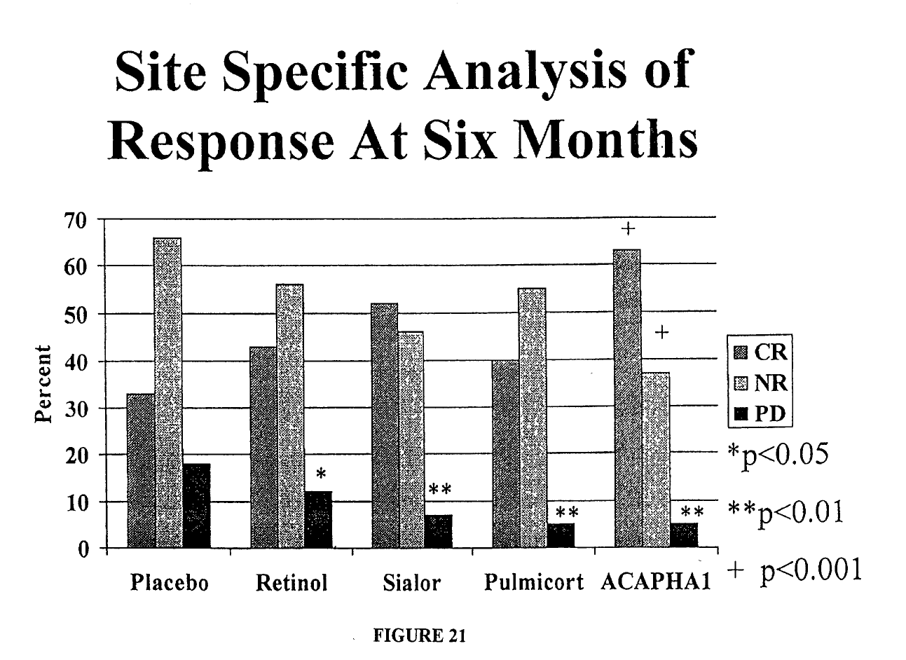

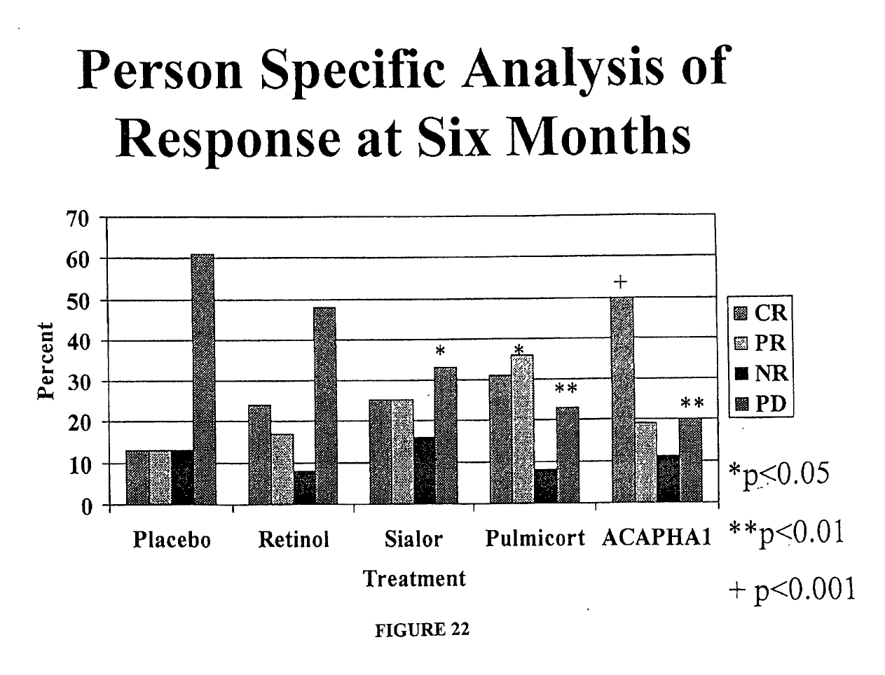

With reference to FIGS. 21 and 22, at six months, the complete regression rate of areas with dysplasia was significantly higher after taking ACAPHA 1 than placebo (63% versus 33%, P<0.001). The NR and PD rates were also significantly lower (NR 37% versus 66%, P<0.001; PD 5% versus 18%, P=0.008) There were 8 new lesions that were moderate/severe dysplasia and 32 new lesions with mild dysplasia in the placebo group but only 1 new lesion with mild dysplasia in the ACAPHA 1 group. This response is higher than any known chemopreventative agent that has been tested in Phase II/III clinical trials thus far.

In the person specific analysis, the CR rate was 50% in those who took ACAPHA 1 versus 13% in those who were on placebo (p<0.001). The PD rate was also significantly lower in those who were on ACAPHA 1 (20% versus 61%, P=0.003).

Multiple logistic regression analysis was used to determine the simultaneous effects of age, gender, smoking intensity (pack-years) and the effect of treatment on the CR rate. ACAPHA 1 had a strong effect on the CR rate (P=0.001). Gender had a strong association with CR (P=0.004) suggesting that women had, on average 6.5 times higher odds of CR (95% CI=1.8 to 23.1 times). None of the remaining variables had a significant association with CR.

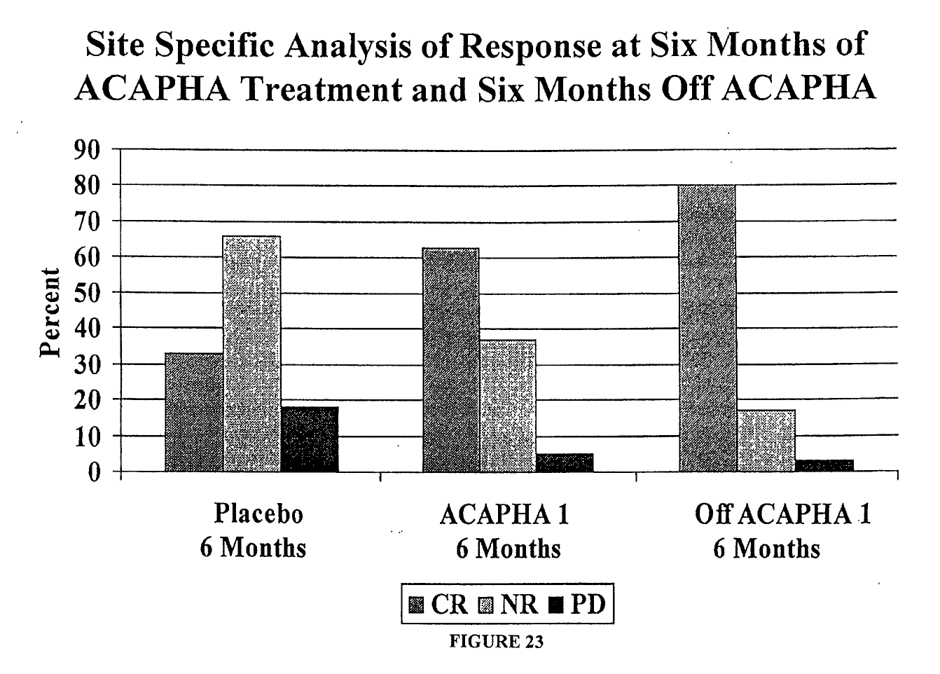

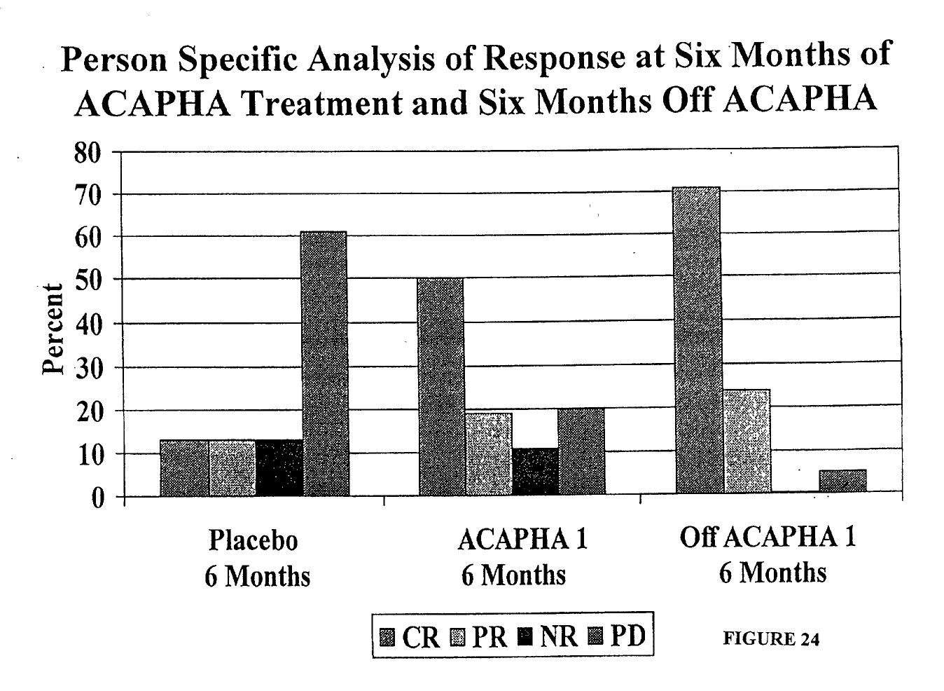

Seventeen subjects were available for the twelve month follow-up assessment after being off ACAPHA for six months. FIG. 23 shows, on a site-by-site analysis, the CR, NR and PD rates were 80%, 17% and 3% respectively. FIG. 24 shows, on a person specific analysis, the CR, PR and PD rates were 71%, 24% and 5% respectively. Thus, the effects of ACAPHA 1 were sustained even after the participants were taken off the treatment for six months.

All the participants tolerated the ACAPHA 1 well. There was no symptomatic adverse event. Sub-clical elevation of AST was observed in 7 of the 20 participants. One of them had preexisting hepatitis C. Alcohol consumption was thought to be the reason for the AST elevation in another participant. The AST level returned to normal after reducing the dose of ACAPHA 1 to 8 tablets once daily in 4 subjects and discontinuation of the ACAPHA 1 in two subjects. The seventh subject was able to complete the six months course at the full dose with mild elevation of AST only. LD levels were also elevated in a number of patients. The liver enzyme results from some patients are shown below in Table 16. Following these results it is suggested that the ACAPHA 2 or 3 formulation be considered for further application as these formulations are less toxic but are as therapeutically effective.

The results show that in both the subject specific and the lesion specific analyses, there was a significantly better complete response rate in the treatment group than in the control group after 6 months of ACAPHA 1 treatment. There was also a significant reduction in the progression rate in the lesion specific analysis when the ACAPHA 1 treatment group was compared to the placebo group. This response is better than any known chemopreventive agent that has been tested thus far. FIGS. 21 and 22 compare the efficacy of ACAPHA 1 with placebo, Retinol (Vitamin A), Sialor (Anetholtrithione) and Pulmicort™ (Inhaled budesonide). As indicated above, the proportion of subjects having a complete response (CR) was significantly higher than other potential chemopreventive agents. Furthermore, the effects of ACAPHA were sustained even after the participants were taken off the treatment for six months.

TABLE 16
ACAPHA 1: Liver Results

| | Month | | | | | | | | |
|---|---|---|---|---|---|---|---|---|---|
| Patient | 1 | 2 | 3 | 0.125 | 4 | 0.166667 | 4B | 4C | 5 |
| 1 | AST* 20 | AST 27 | AST 24 | AST | AST 23 | AST | AST | AST | AST. 28 |
| | LD** 103 | LD 122 | LD 127 | LD | LD 126 | LD | LD | LD | LD 117 |
| 2 | AST 27 | AST 26 | AST 27 | AST | AST 22 | AST | AST. | AST | AST 24 |
| | LD 150 | LD 149 | LD 168 | LD | LD 155 | LD | LD | LD | LD 168 |
| 3 | AST: 34 | AST | AST +44 | AST | AST: +58 | AST | AST | AST. | AST +68 |
| | LD 140 | LD | LD 175 | LD | LD 589 | LD | LD | LD: | LD 844 |
| 4 | AST 20 | AST 23 | AST: 24 | AST | AST 20 | AST: | AST. | AST: | AST. 22 |
| | LD 123 | LD: 128 | LD 128 | LD | LD 142 | LD | LD | LD | LD 124 |
| 5 | AST 29 | AST 68 | AST 29 | AST | AST 27 | AST | AST | AST | AST. 32 |
| | LD 132 | LD 170 | LD. 140 | LD. | LD 145 | LD | LD | LD | LD 175 |
| 6 | AST 20 | AST +11 | AST +16 | AST | AST +18 | AST | AST. | AST. | AST 20 |
| | LD 369 | LD 138 | LD 138 | LD | LD 142 | LD | LD | LD | LD. 154 |
| 7 | AST 16 | AST 18 | AST | AST | AST 17 | AST | AST | AST. | AST 21 |
| | LD 147 | LD 174 | LD | LD | LD 168 | LD | LD | LD. | LD 181 |
| 8 | AST 28 | AST 30 | AST 33 | AST: | AST +43 | AST. | AST | AST | AST +191 |
| | LD 135 | LD. 150 | LD 149 | LD | LD. 157 | LD | LD | LD. | LD +222 |
| 9 | AST 20 | AST. 21 | AST: 31 | AST | AST. +45 | AST: +45 | AST | AST | AST. +48 |
| | LD 103 | LD. 117 | LD 127 | LD: | LD. 133 | LD 133 | LD | LD | LD. 124 |
| 10 | AST 23 | AST 25 | AST 25 | AST | AST +301 | AST +131 | AST +92 | AST +73 | AST +88 |
| | LD 118 | LD 110 | LD 123 | LD | LD 193 | LD 164 | LD 185 | LD 493 | LD 151 |
| 11 | AST 20 | AST 22 | AST 27 | AST: | AST 29 | AST | AST | AST | AST: 32 |
| | LD 97 | LD. 116 | LD 134 | LD | LD. 110 | LD | LD | LD. | LD 121 |
| 12 | AST 22 | AST 19 | AST +18 | AST | AST +17 | AST | AST | AST | AST: +17 |
| | LD 148 | LD. 168 | LD 173 | LD | LD 158 | LD | LD | LD. | LD 158 |
| 13 | AST 28 | AST 30 | AST 25 | AST | AST 25 | AST | AST | AST: | AST 24 |
| | LD 161 | LD 151 | LD 155 | LD | LD 136 | LD | LD | LD | LD 146 |
| 14 | AST 21 | AST 25 | AST 27 | AST | AST 26 | AST | AST | AST | AST 24 |
| | LD 154 | LD 144 | LD 154 | LD | LD 148 | LD. | LD | LD. | LD 138 |
| 15 | AST +17 | AST +18 | AST 34 | AST | AST +15 | AST | AST | AST. | AST. 19 |
| | LD 130 | LD 147 | LD. 145 | LD. | LD: 139 | LD. | LD | LD | LD. 154 |
| 16 | AST 30 | AST. 30 | AST 30 | AST | AST. 31 | AST | AST | AST. | AST: 31 |
| | LD 163 | LD: 189 | LD 189 | LD | LD. 158 | LD. | LD | LD | LD. 158 |
| 17 | AST. 19 | AST 21 | AST | AST | AST 18 | AST | AST. | AST | AST +15 |
| | LD. 113 | LD 132 | LD | LD | LD 124 | LD | LD | LD | LD 130 |
| 18 | AST 24 | AST 25 | AST. 28 | AST | AST 27 | AST. | AST | AST | AST: 27 |
| | LD 137 | LD. 140 | LD 147 | LD | LD 147 | LD. | LD. | LD. | LD. 150 |
| 19 | AST 21 | AST +16 | AST +18 | AST | AST +17 | AST | AST: | AST | AST +16 |
| | LD 113 | LD 120 | LD 129 | LD | LD 125 | LD | LD | LD | LD 143 |
| 20 | AST +43 | AST | AST +161 | AST +113 | AST +65 | AST | AST | AST | AST +179 |
| | LD 141 | LD 146 | LD 148 | LD 478 | LD 132 | LD | LD | LD. | LD. +209 |

Month

| | | | | | | | | | | 9-1YR |
|---|---|---|---|---|---|---|---|---|---|---|
| Patient | 0.208333 | 5B | 6 | 0.25 | 7 | 0.291667 | 7B | 7C | 8 | F/U |
| 1 | AST | AST | AST 29 | AST | AST +93 | AST +171 | AST +295 | AST +73 | AST | AST 22 |
| | LD | LD | LD 120 | LD | LD 138 | LD | LD 575 | LD 386 | LD | LD 109 |
| 2 | AST | AST | AST 20 | AST | AST 24 | AST | AST | AST | AST | AST. 27 |
| | LD. | LD | LD 157 | LD. | LD 158 | LD | LD | LD | LD | LD 187 |
| 3 | AST +72 | AST. | AST +59 | AST | AST +73 | AST | AST | AST | AST | AST. +71 |
| | LD 591 | LD | LD 521 | LD | LD 184 | LD | LD | LD | LD. | LD 204 |
| 4 | AST: | AST. | AST. 20 | AST | AST 20 | AST | AST | AST | AST | AST 22 |
| | LD. | LD | LD 123 | LD | LD 117 | LD | LD | LD | LD | LD 161 |
| 5 | AST | AST | AST. +146 | AST +84 | AST. +51 | AST 36 | AST | AST | AST | AST 27 |
| | LD | LD | LD +212 | LD: 533 | LD 167 | LD. 157 | LD | LD | LD | LD 165 |
| 6 | AST | AST | AST: 19 | AST. | AST. +18 | AST | AST | AST. | AST | AST 22 |
| | LD | LD | LD. 131 | LD. | LD 140 | LD | LD | LD | LD | LD: 147 |
| 7 | AST | AST | AST: 20 | AST. | AST 21 | AST | AST | AST | AST | AST +18 |
| | LD | LD | LD: 166 | LD | LD 189 | LD. | LD | LD | LD. | LD. 192 |
| 8 | AST +542 | AST +262 | AST +68 | AST +111 | AST +234 | AST +220 | AST +56 | AST 29 | AST | AST 28 |
| | LD +1060 | LD +666 | LD 167 | LD 521 | LD +218 | LD 641 | LD 479 | LD 125 | LD: | LD. 139 |
| 9 | AST: | AST. | AST +60 | AST | AST +102 | AST | AST. | AST | AST | AST 24 |
| | LD. | LD | LD 131 | LD. | LD 151 | LD | LD | LD | LD | LD 112 |
| 10 | AST. | AST. | AST +49 | AST. | AST +70 | AST +50 | AST | AST: | AST. | AST. 19 |
| | LD | LD | LD. 120 | LD | LD. 137 | LD 415 | LD | LD | LD | LD 123 |
| 11 | AST | AST | AST 25 | AST | AST. 25 | AST: | AST. | AST: | AST: 22 | AST: 33 |
| | LD. | LD | LD: 114 | LD | LD 117 | LD | LD | LD: | LD. 113 | LD 118 |
| 12 | AST | AST. | AST: +18 | AST | AST 21 | AST: | AST. | AST. | AST: | AST 22 |
| | LD: | LD | LD: 155 | LD | LD. 176 | LD: | LD | LD | LD | LD 532 |
| 13 | AST | AST | AST: 28 | AST | AST 22 | AST | AST | AST | AST: | AST 27 |
| | LD | LD | LD 147 | LD: | LD 123 | LD. | LD. | LD | LD: | LD 155 |
| 14 | AST | AST. | AST 25 | AST | AST 19 | AST. | AST | AST. | AST: | AST: 30 |
| | LD. | LD. | LD 167 | LD: | LD: 454 | LD | LD: | LD. | LD | LD.178 |
| 15 | AST. | AST: | AST 23 | AST | AST +18 | AST. | AST | AST: | AST: | AST +17 |
| | LD: | LD: | LD 127 | LD. | LD 102 | LD. | LD. | LD | LD: | LD 148 |
| 16 | AST | AST. | AST: 26 | AST | AST 29 | AST. | AST. | AST. | AST: | AST 31 |
| | LD. | LD. | LD. 150 | LD | LD: 162 | LD: | LD: | LD. | LD. | LD 168 |
| 17 | AST | AST | AST 19 | AST | AST. 17 | AST | AST: | AST: | AST: | AST. 20 |
| | LD | LD | LD: 129 | LD | LD. 401 | LD | LD: | LD. | LD. | LD: 413 |
| 18 | AST: | AST | AST. 20 | AST | AST: 27 | AST | AST | AST: | AST | AST 31 |
| | LD. | LD: | LD: 132 | LD. | LD. 154 | LD | LD: | LD | LD: | LD: 153 |
| 19 | AST. | AST | AST: +15 | AST | AST 20 | AST | AST | AST. | AST | AST |
| | LD | LD | LD 133 | LD | LD 125 | LD: | LD | LD. | LD: | LD: |
| 20 | AST +323 | AST | AST +83 | AST +50 | AST 32 | AST | AST | AST | AST. | AST 20 |
| | LD +687 | LD | LD 136 | LD 361 | LD 139 | LD | LD: | LD: | LD | LD: 129 |

*AST = U/L
**LD = U/L

### Example 5

### Effect of ACAPA 1 on Chronic Atrophic Gastritis

Chronic atrophic gastritis is a common disease in middle and old age. The etiology and mechanism of the disease is still unclear. As a result, there is still no specific effective therapy and current treatment is mainly directed at treatment of symptoms.

In chronic atrophic gastritis, gastric mucosal epithelial gland metaplasia and a typical hyperplasia are characteristic pathological features, which are also closely associated with the development of gastric carcinoma. Some scientists have even considered that these features are a precancerous pathological change.

Intestinal gland metaplasia refers to the presence of intestinal glandular epithelium in the gastric mucosa. Based on the amount present, the disease can be classified into mild, moderate and severe categories.

Atypical hyperplasia of gastric mucosal epithelium is also referred to as gastric dysplasia. The main features include a typical morphology of the cells, irregular architecture and a typical cellular distribution. According to the degree of tissue involvement the changes can also be classified into mild, moderate and severe categories. Due to the absence of specific effective drugs to improve or cure this condition, surgery is usually recommended to prevent the development of cancer in severe cases of dysplasia. Development of a new drug that can improve or eradicate intestinal gland metaplasia and dysplasia should greatly reduce the incidence of gastric carcinoma. In this example, the effect of ACAPHA 1 on chronic atrophic gastritis was investigated.

A total of 26 subjects were used in this study conducted in China. All patients were diagnosed with chronic atrophic gastritis and had either intestinal gland epithelium metaplasia or dysplasia. Selection of patients was carried out according to strict international diagnostic criteria. The severity of gastric mucous gland epithelium metaplasia and dysplasia was also assessed according to strict international criteria.

Before selection, an experienced endoscopist examined the gastric lining of each patient and obtained gastric mucosal biopsy samples at defined sites. An experienced pathologist then examined histological sections of the biopsy samples. Cases that fulfilled the selection criteria were selected for the study. Detailed case histories were then recorded for each patient and WBC, kidney and liver function tests were performed.

Patients who were admitted into the study were told to take 3 ACAPHA 1 tablets, twice a day for 3 months. All other medications for the treatment of chronic atrophic gastritis were stopped. At the end of the treatment period, adverse reactions and side effects caused by ACAPHA 1 and clinical changes were recorded. WBC, liver and kidney function tests were once again performed. The same endoscopist examined the gastric lining of the patients and recorded any observable changes. A repeat sampling of gastric mucosal biopsies were performed in the same sites as before and the same pathologist examined histological sections from the biopsies. Finally, an independent experienced pathologist reexamined each histological section to confirm the accuracy of the findings.

None of the study patients with chronic atrophic gastritis experienced adverse reactions or side effects after taking ACAPHA 1. In addition, there were no significant changes in the WBC, liver and kidney function tests after ACAPHA 1 therapy. In 16 cases (61.5%), clinical symptoms of abdominal pain, belching (eructation), and abdominal distention significantly improved after 1 month of ACAPHA 1 therapy After 2 months, 22 cases (85.4%) showed significant improvement of clinical symptoms and after 3 months, only one patient did not have significant improvement Therefore, the clinical symptom improvement rate was 96.2% after three months.

Comparison of endoscopic examinations before and after ACAPHA 1 treatment indicated that 7 cases with focal necrosis had significant improvement or disappearance of lesion following treatment. There were no significant changes in other features of chronic atrophic gastritis revealed by endoscopy.

Histopathological examination of biopsy sections before and after ACAPHA 1 treatment revealed significantly more than the endoscopic examinations. It was found that 7 cases of chronic atrophic gastritis regressed to chronic superficial gastritis. In particular, out of 12 cases of severe intestinal gland epithelium metaplasia, 5 regressed to moderate or mild forms of the metaplasia and 2 no longer showed any features of severe metaplasia. Out of 9 cases with moderate intestinal glandular epithelium metaplasia, 3 regressed to mild metaplasia and the remaining 6 no longer showed any features of moderate metaplasia. Finally, in all 5 cases of mild intestinal glandular epithelium metaplasia, all abnormal features disappeared after ACAPHA 1 treatment. In the cases of severe a typical hyperplasia, 4 cases regressed to mild a typical hyperplasia and in 2 cases the pathological changes disappeared. In all 8 cases with moderate a typical hyperplasia and 12 cases with mild a typical hyperplasia the abnormal features disappeared after ACAPHA 1 treatment.

The results from this study indicate that ACAPHA 1 has no toxic side effects toward WBC, kidney and liver functions and is effective against chronic atrophic gastritis and intestinal gland epithelium metaplasia and dysplasia.

### Example 6

### Effect of ACAPHA 1 on Helicobacter pylori Infection Associated Polyp Type Gastritis

Polyp type gastritis is a special form of chronic gastritis. It can be present as the only problem, or it can exist with other gastric disorders. The special feature is the presence of pathological umbilicus shaped swellings of the gastric mucosa. In China, it comprises about 15 of chronic gastritis and 85% of these patients have H. pylori (HP) infection.

H. pylori is important in the etiology of gastric cancer. It has recently been recognized by the International Agency for Research on Cancer as a human carcinogen. HP is assumed to cause gastric cancer indirectly because it provokes gastritis, which is a precursor of gastric atrophy, metaplasia and dysplasia. In the following example, the effect of ACAPHA 1 on HP infection associated polyp type gastritis was investigated.

A total of 16 patients, ages ranging from 35 to 62 years old, were used in this study. Disease duration ranged from 4 to 12 months, with a mean of 7 months. Selection criteria of polyp type gastritis was based on diagnostic criteria and HP infection was tested using urease test and biopsy sections.

An experienced endoscopist examined each patient prior to selection. Detailed descriptions were made during the endoscopic assessment and photos were taken. In addition, a minimum of 2 mucosal specimens in areas with pathological changes were collected and examined by an experienced pathologist. Patients that fulfilled the criteria for polyp type gastritis and tested positive for HP infection were included in the study group. The detailed clinical condition of the test subjects were then recorded and WBC counts and liver and kidney function tests were performed.

Patients in the study group were instructed to take 8 tablets of ACAPHA 1, twice a day for 3 months (a total of 16 tablets/day). During the treatment, all other medications used for treatment of gastric conditions were stopped. At the end of the treatment period, side effects of ACAPHA 1 and changes in the disease states were recorded. WBC, liver and kidney function tests were repeated. The same endoscopist repeated the gastric endoscopic examinations, recording any observable changes and taking photos. Biopsy samples were taken in the same mucosal areas as before and the same pathologists assessed the histological sections. The presence of HP infection was again tested.

None of the 16 patients had adverse reactions or side effects during treatment with ACAPHA 1. There were also no significant differences in the WBC, liver and kidney function tests following ACAPHA 1 treatment. Among the treated patients, 13 cases (81.3%) had obvious improvement in the original clinical symptoms of gastric distention, intestinal pain, heart burn due to hypersecretion of gastric acid and vomiting after 1 month of ACAPHA 1 treatment. After 2 months of treatment, the original clinical symptoms disappeared in these patients. After 3 months of treatment, only 2 cases showed no significant improvement in clinical symptoms. The efficacy rate as assessed by clinical symptoms was 87.5%.

After 3 months of ACAPHA 1 treatment, endoscopic examination revealed that in 12 of the 16 cases, the pathological features of mucosal umbilicus type swelling had totally disappeared. Histopathological assessment showed that there was regression from polyp type gastritis to a mild form of superficial gastritis. In addition, 11 cases tested negative for HP infection. There were 4 cases that did not respond to treatment but after an additional 3 months of ACAPHA 1 treatment, 3 patients no longer showed gastric mucosal umbilicus type swelling. Histopathological assessment of these patients showed a regression to a mild form of superficial gastritis. In 2 of the 4 cases, HP infection tested negative. The overall efficacy rate of ACAPHA 1 treatment as assessed by gastric endoscopy and histopathology was 93.8%. The HP infection curative rate was 81.3%%.

The results showed that ACAPHA 1 was an effective treatment for polyp type gastritis as measured by clinical assessment, endoscopic examination and histopathological assessment. In addition, ACAPHA 1 was effective in eradicating HP infection. The eradication rate of HP infection by ACAPHA 1 is similar to the rate achieved by the internationally recognized standard treatment of HP using a double drug regimen, consisting of bismuth compound and an antibiotic, or bismuth-based triple therapy. Finally, there were no adverse effects associated with ACAPHA 1 treatment.

### Example 7

### Effect of ACAPHA 1 on Oral Leukoplakia

Oral leukoplakia is a common oral mucosal precancerous lesion that often progresses to oral cancer. Presently, patients with oral leukoplakia are monitored for disease progression and the lesions are surgically removed when they become cancerous The problem with this form of treatment is that tumor recurrence is quite common. More recently, the efficacy of laser treatment, cryosurgery and hyperthermia therapies have been debated. Chemotherapy is still at the investigation stage but problems relating to disease recurrence after discontinuation of drug treatment and toxic side effects have been found.

In this example the effect of ACAPHA 1 on an animal model of chemically induced oral leukoplakia and clinically in patients with oral leukoplakia was investigated.

A total of 43 Syrian golden hamsters at 4 to 8 weeks old were treated with 0.5% DMBA on the oral mucous membrane 3 times per week for 6 weeks to induce oral leukoplakia. The hamsters were then spilt into treatment and control groups. The treatment group was given 2 ACAPHA 1 tablets a day for 10 weeks, while the control group was not given anything after DMBA induction.

Biopsies were performed on the hamsters following 3 weeks of DMBA treatment, 10 weeks of ACAPHA 1 treatment, and 8 weeks post ACAPHA 1 treatment as a follow up. Tissue samples were fixed in formalin, processed, and then stained with hematoxylin and eosin. Histological changes were classified according to standard Smith and Pindborg classification.

After three weeks of DMBA treatment, histological changes in the mucous membrane cavity of the animals included 58.8% with mild dysplasia and 46.2% with hyperplasia. After 10 weeks of ACAPHA 1 treatment, oral cancer rate was 35.3% in the treatment group compared to 96.2% in the control group. Follow up observation at 8 weeks showed that the ACAPHA 1 treatment group had a cancer rate of 47.1% compared to 100% in the control group.

A total of 66 patients with oral leukoplakia were randomized into 2 groups consisting of 36 patients in the treatment group and 30 patients in the control group. The ages of the patients ranged from 38 to 70. Oral leukoplakia was assessed according to WHO criteria. All other possible causes that could induce oral mucosal changers, such as tobacco, poor dental hygiene, and bacterial infections were eliminated.

The treatment group was further divided into those with mild leukoplakia and those with severe leukoplakia. The mild leukoplakia group was given 3 ACAPHA 1 tablets, 3 times daily (9/day) for 4 months. The severe leukoplakia group was given 6 ACAPHA 1 tablets, 3 times daily (18/day) for 4 months. The control group was given a placebo treatment for 4 months.

In the treatment group, 8 patients displayed marked improvement, 19 showed improvement and 9 showed no change. This demonstrated an improvement rate of 75%. In the control group, 5 patients showed improvement and the remaining 25 showed no change. The improvement rate for the control group was only 16.7%.

During treatment with ACAPHA 1, it was observed that 2 patients had stomach discomfort and 1 patient complained of a dry mouth. However, no significant changes were seen in liver function tests after therapy. These results show that ACAPHA 1 is an effective treatment for oral leukoplakia.

### Example 8

### The Effect of ACAPHA 1 on Oral Lichen Planus

Oral Lichen Planus (OLP) is a chronic inflammatory condition associated with the immune system. Some reports have suggested that it has a tendency to become cancerous. Presently there is no effective treatment for this condition. In this example, the effects of ACAPHA 1 on patients with OLP were investigated.

Ten patients with confirmed diagnoses of OLP and unresponsive to other forms of treatment were used in this study. One month prior to ACAPHA 1 treatment, all patients stopped taking medication that may affect the immune system. ACAPHA 1 was given at a dose of 3 tablets, 3 times a day for 4 to 12 months. The patients were given oral examinations every 2 months and had serum and urine tests for SGPT and BUN. Following ACAPHA 1 treatment, OLP in the patients was monitored for another 4 to 7 months.

The overall effective rate was 90%, with 10% cured, 40% showing marked improvement, 40% showing improvement and 10% showing no change. The liver and kidney functions remained normal during the study indicating that ACAPHA 1 did not have any detectable side effects.

The results suggest that ACAPHA 1 is an effective treatment for OLP. In view of the fact that the treatment is simple and inexpensive and long term treatment did not affect liver and kidney functions, this would suggest that ACAPHA 1 has potential as a treatment for OLP and to prevent it from progressing to oral cancerous lesions

### Example 9

### Effect of ACAPHA on Human Prostate Cancer Cell Growth

Prostate cancer is an increasing public health problem among men in the United States. If caught at an early stage, prostate cancer often responds to orchitectomy or antiandrogen therapy. However, over time residual androgen-insensitive cells will recolonize, expand and establish a hormone-resistant state, leading to a recurrence of the prostate cancer. In the following example the effect of ACAPHA on androgen responsive and androgen refractory human prostate cell lines was investigated. The results showed that ACAPHA is an effective inhibitor of human prostate cell growth.

Human prostate cell lines (DU-145, LNCAP, and PC-3) were obtained from American Type Culture Collection (Rockville, Md.). These cell lines were derived from various sites to which primary tumor had metastasized. The JCA-1 cells were established at the New York Medical College from the primary prostate cancer site prior to the administration of hormone. Androgen-responsive LNCaP cells may be representative of latent prostate cancer, whereas androgen-refractory JCA-1, PC-3, and DU-145 cells are considered to better represent advanced prostate cancer. Cells were maintained in RPMI 1640 media containing 2 mM glutanine, 100 units/ml penicillin, 100 μg/ml streptomycin, and supplemented with 10% heat-inactivated fetal calf serum.

ACAPHA solutions for the in vitro studies were prepared by stirring ACAPHA powder or tablets in 70% ethanol (340 mg/ml) at 150 rpm for one hour at room temperature. Insoluble material was removed by centrifugation. The ethanolic extracts were sterilized by passing through 0.22 μm filters and kept in aliquots in a refrigerator. Final concentrations of ACAPHA used for different experiments were prepared by diluting the stock with RPMI1640. Control cultures received the carrier solvent. Two different lots of ACAPHA 1 tablets and three different formulations of ACAPHA in powder form ACAPHA 1, ACAPHA 2 and ACAPHA 3 were prepared in an identical manner.

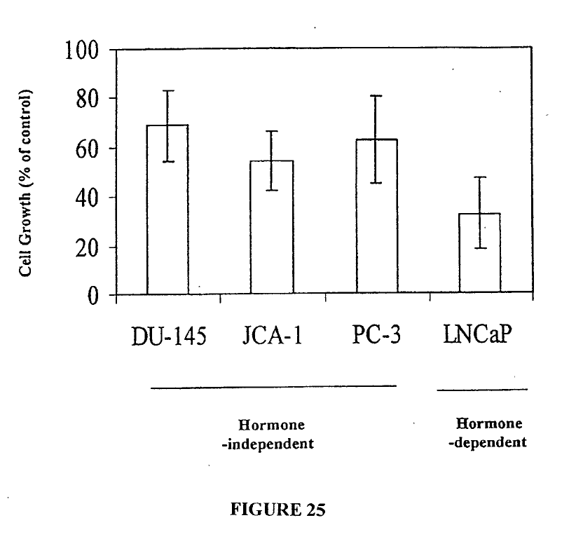

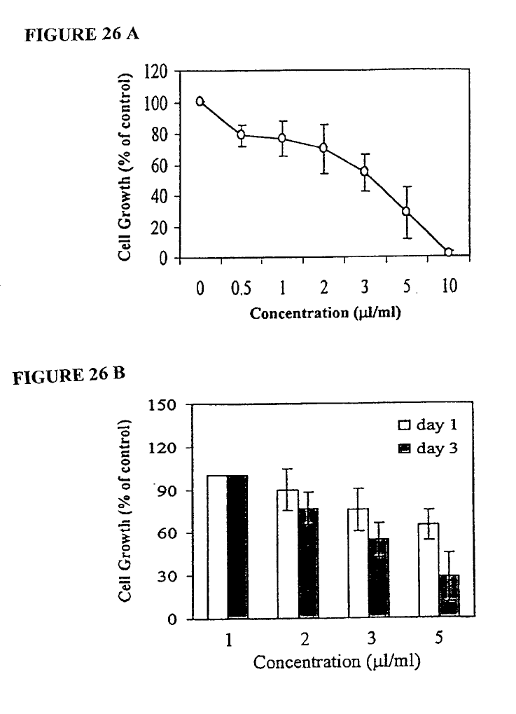

Effect of ACAPHA on Growth of Prostate Cancer Cells. Prostate cancer cell lines were treated with different concentrations of ACAPHA and after 24, 48 and 72 hours, cells were harvested. Cell number was determined by hemocytometer and cell viability was assessed by trypan dye exclusion assay. Ethanolic extracts of ACAPHA 1 inhibited cell proliferation in both hormone-dependent and hormone-independent prostate cancer cells. At 3 μg/ml of ACAPHA 1, hormone-dependent LNCap cells was more sensitive to the treatment than hormone-refractory cell lines. The growth suppression is shown as follows: LNCAP (67.4%)>JCA-1 (45.7%)>PC-3 (37.5%) >DU-145 (31.1%) (FIG. 25). Further studies showed that growth suppression of JCA-1 cells was both concentration and time dependent. This cell line has been shown to be refractory to inhibition by resveratrol and other agents currently used for treatment of prostate cancer. With 3 μg/ml of ACAPHA 1 treatment it reached its IC₅₀ and dramatic growth inhibition accompanying with decreasing in cell viability was found at 5 and 10 μg/ml of ACAPHA 1 (FIG. 26A) and more significant effect was found in day 3 of treatment (FIG. 26B). The study demonstrates that ACAPHA 1 has potent inhibitory activity on both androgen-responsive and androgen-refractory human prostate cells. Therefore, ACAPHA 1 may be useful for patients who have failed to respond to conventional therapy.

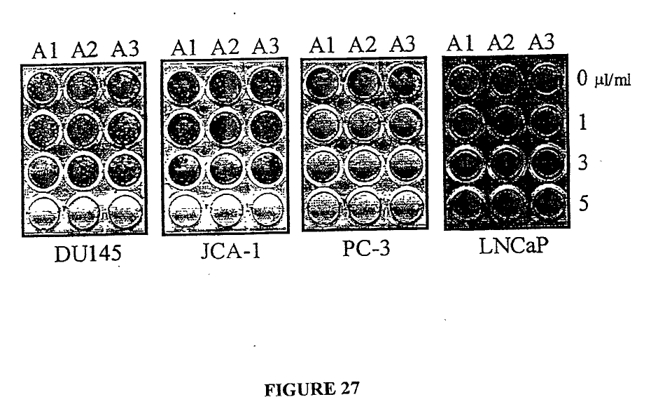

Effect of ACAPHA on Clonogenicity of Prostate Cancer Cells. The growth suppressive effect of three different formulations (compositions) of ACAPHA was tested with the clonogenic assay. FIG. 27 shows that dose-dependent inhibition was observed whereas formulation variation was not seen. ACAPHA1, 2 and 3 effectively suppressed colony formation against all four human prostate cancer cell lines.

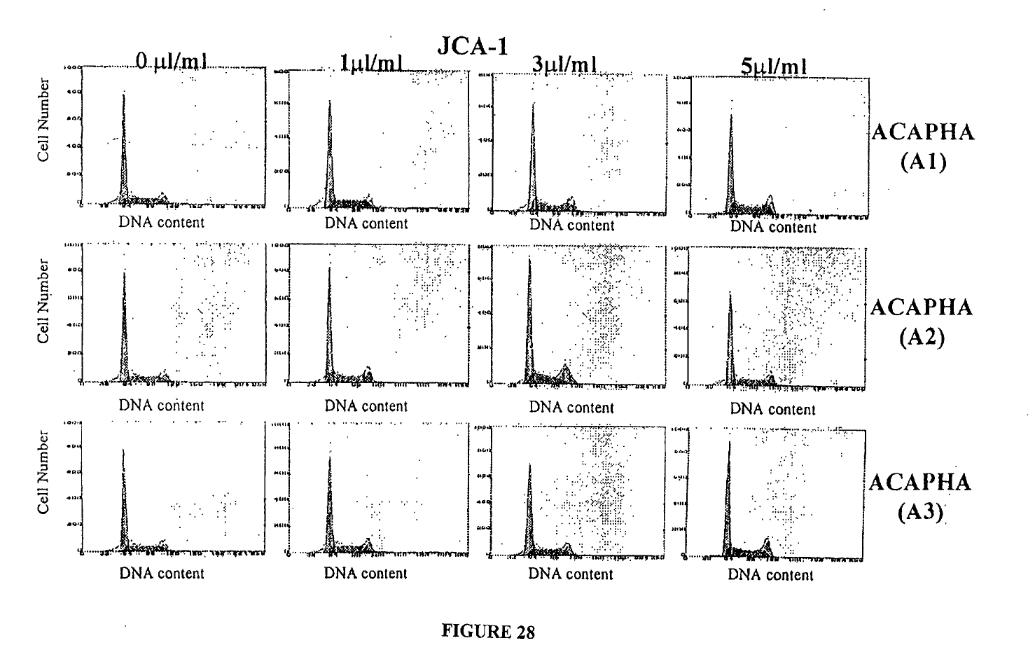

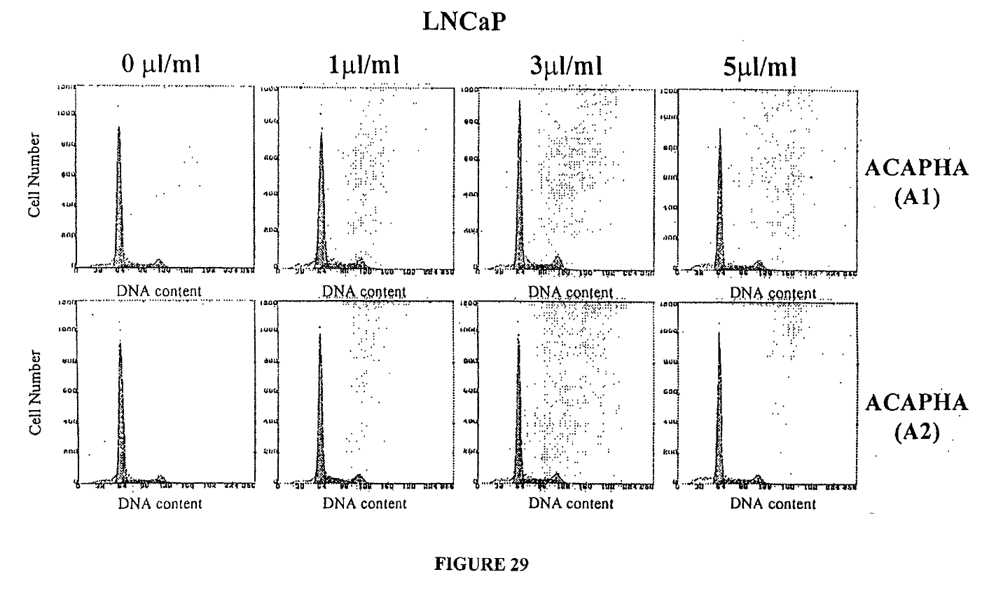

Effect of ACAPHA on Cell Cycle Progression. Cultures were exposed to varying concentrations of ACAPHA 1 (peripheral blood lymphocytes) or ACAPHA 1, 2 and 3 (JCA-1 and LNCAP cells). Cells were harvested, stained with DAPI fluorochrome and analyzed with a flow cytometer. ACAPHA 1 did not have a significant effect on cell cycle distribution of unstimulated and mitogen-stimulated peripheral blood lymphocytes. These results are presented in Tables 17 and 18. FIGS. 28 and 29 similarly show that ACAPHA 1 did not have a significant effect on cell cycle distribution of androgen-refractory human JCA-1 and androgen-responsive LNCAP cell lines. Further results are also shown in Tables 19 and 20. Using Jurkat (human T cell leukemia cell line) T cells and JCA-1 prostate cancer cells, ACAPHA 1 displayed differential growth suppressive effects on these two cell types, exerting minimal effects on T cell growth while substantially reducing growth of prostate cancer cells. This data suggests that ACAPHA has no apparent toxic effects. It was also demonstrated that the growth suppressive effect is unrelated to changes in p53 and p21 expression (Table 21).

TABLE 17
Distribution of the various phases of the cell cycle

| | Time | ACAPHA 1 | Cell Cycle Distribution | | |
|---|---|---|---|---|---|
| PBL | (hr) | (μl/ml) | G1 | S | G2/M |
| −PHA | 48 | 0 | 93.6 | 0.7 | 5.6 |
| −PHA | 48 | 1 | 95.3 | 0.5 | 4.2 |
| −PHA | 48 | 3 | 92.8 | 0.8 | 6.4 |
| −PHA | 48 | 5 | 96.6 | 0.7 | 2.7 |
| +PHA | 48 | 0 | 62.4 | 30 | 7.6 |
| +PHA | 48 | 1 | 63.1 | 29.6 | 7.3 |
| +PHA | 48 | 3 | 71.2 | 19.7 | 9.1 |
| +PHA | 48 | 5 | 73.8 | 19.5 | 6.7 |

TABLE 18
Distribution of the various phases of the cell cycle

| | Time | ACAPHA 1 | Cell Cycle Distribution | | |
|---|---|---|---|---|---|
| PBL | (hr) | (μl/ml) | G1 | S | G2/M |
| −PHA | 72 | 0 | 91.9 | 1.6 | 6.5 |
| −PHA | 72 | 1 | 91.7 | 1.4 | 6.9 |
| −PHA | 72 | 3 | 90.8 | 1.7 | 7.5 |
| −PHA | 72 | 5 | 89.7 | 1.8 | 8.5 |
| +PHA | 72 | 0 | 60.6 | 29.4 | 10 |
| +PHA | 72 | 1 | 53.8 | 35.3 | 10.8 |
| +PHA | 72 | 3 | 52.8 | 36.1 | 11.1 |
| +PHA | 72 | 5 | 56.8 | 31.8 | 11.4 |

TABLE 19
Shows the quantitation of the effects of various concentrations of
ACAPHA 1 (A1), ACAPHA 2 (A2) and ACAPHA 3 (A3) respectively
on changes in the distribution of cell cycle phase progression in the
androgen-refractory human prostate JCA-1 cell line. Cells were treated
with ACAPHA for 72 hours.

| | Treatment | | | | |
|---|---|---|---|---|---|
| JCA-1 | (μl/ml) | G1 | S | G2/M | Apoptosis |
| control | | 61.3 | 30.2 | 8.5 | |
| ACAPHA-A1 | 1 | 60.9 | 32.9 | 6.1 | |
| ACAPHA-A1 | 3 | 54.8 | 38.2 | 7 | |
| ACAPHA-A1 | 5 | 50.3 | 41.1 | 8.6 | |
| ACAPHA-A2 | 1 | 55.4 | 35.6 | 9 | |
| ACAPHA-A2 | 3 | 55.9 | 26 | 18.1 | |
| ACAPHA-A2 | 5 | 58 | 31.7 | 10.2 | |
| ACAPHA-A3 | 1 | 59.2 | 31.7 | 9.1 | |
| ACAPHA-A3 | 3 | 61.9 | 28.9 | 9.2 | |
| ACAPHA-A3 | 5 | 52.6 | 34.9 | 12.4 | |

TABLE 20
The Quantitation of the effects of various concentrations of ACAPHA
1 (A1) and ACAPHA 2 (A2) respectively on changes in the distribution of
cell cycle phase progression in the androgen-responsive human prostate
LNCaP cells. Cells were treated with ACAPHA for 72 hours.

| | Treatment | | | | |
|---|---|---|---|---|---|
| LNCaP | (μl/ml) | G1 | S | G2/M | Apoptosis |
| control | | 81.7 | 13.6 | 4.7 | |
| ACAPHA-A1 | 1 | 76.4 | 18 | 5.6 | |
| ACAPHA-A1 | 3 | 75.3 | 16.9 | 7.8 | |
| ACAPHA-A1 | 5 | 74.6 | 17.9 | 7.5 | |
| ACAPHA-A2 | 1 | 73.6 | 16.7 | 9.7 | |
| ACAPHA-A2 | 3 | 75 | 17.9 | 7.1 | |
| ACAPHA-A2 | 5 | 75.9 | 18 | 6.2 | |

TABLE 21
Effect of ACAPHA on p523 and p12 Levels

| Treatment | p21 | p53 |
|---|---|---|
| Control | 17.8 | 16.2 |
| ACAPHA-1 (3 μl/ml) | 18.1 | 16.6 |
| ACAPHA-2 (3 μl/ml) | 18.1 | 16.1 |

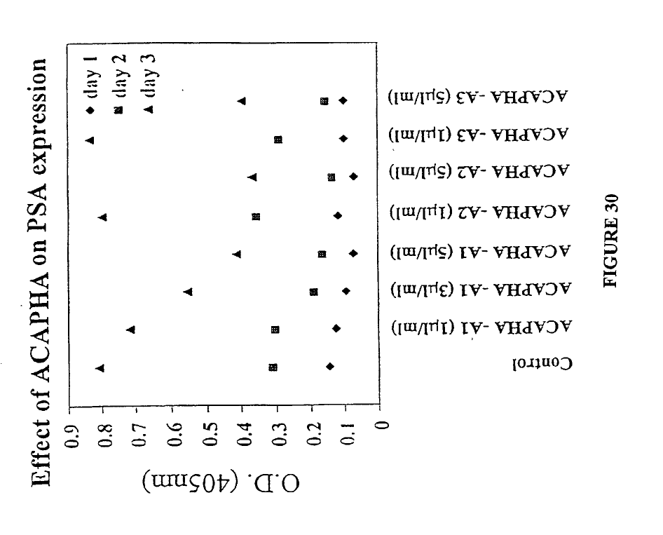

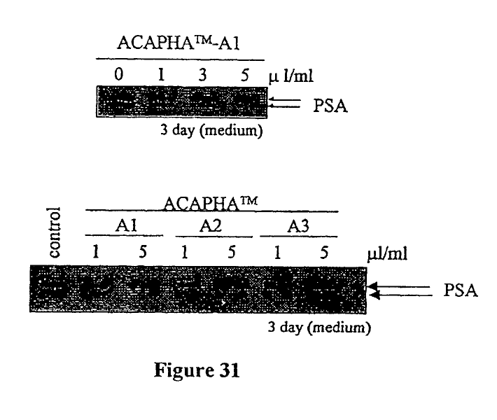

Effect of ACAPHA on Secreted Form of Prostate Specific Antigen (PSA). PSA has been identified as a marker to aid in the diagnosis of prostate cancer and for monitoring their responses to therapy. FIG. 30 illustrates the effect of three formulations of ACAPHA, ACAPHA1 (A1), ACAPH 2 (A2) and ACAPHA 3 (A3), on time and dose-dependent PSA secretion in androgen-responsive LNCaP cells. The figure illustrates that increasing concentrations of ACAPHA significantly inhibited the secretion of PSA in both a dose and time-dependent manner. These results were confirmed by western blot analysis (FIG. 31). Since PSA is a commonly accepted marker for human prostate cancer, these findings suggest that ACAPHA may be potentially useful in alleviating some of the symptoms of prostate carcinogenesis. Since the expression of PSA is downstream of changes in the androgen receptor, cellular androgen receptors and PSA in both control and ACAPHA treated cell extracts were evaluated with immunoblot analysis. Intracellular androgen receptors and PSA expression was down-regulated after treatment with 5 μg/ml of ACAPHA extract (Figure not shown).

In summary, this cell line study indicates that ACAPHA 1 (A1), ACAPHA 2 (A2) and ACAPHA 3(A3) appear to inhibit the growth of both androgen sensitive and androgen refractory prostate cancer cells in vitro. The inhibitory effect does not appear to be related to changes in cell cycle progression, changes in p53 and p21 tumor suppressor gene expression or cellular apoptosis. Further ACAPHA 1 (A1), ACAPHA2 (A2) and ACAPHA 3(A3) inhibited the secretion of PSA tumor marker in vitro. ACAPHA is relatively benign and is not cytotoxic to normal cells. The mechanism of ACAPHA has not yet been elucidated, but it is likely related to genes that are directly or indirectly under the control of NFKB.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

The embodiments of the invention in which an exclusive property of privilege is claimed are defined as follows:

1. A composition comprising a mixture of at least three different herbs selected from the group consisting of: Herb A, Herb B, Herb C, Herb D and Herb E, wherein Herb A is selected from the group consisting of:
Sophora tonkinensis (Sophora subprostrata)
Belamcanda chinensis (L) DC
Scrophularia ningpoensis Hemsl.
Isatis tinctoria L.
Isatis indigotica Fort. and
Baphicacanthus cusia Bremek;
Herb B is selected from the group consisting of:
Polygonum bistorta
Polygonum lapidosum Kitag.
Polygonum viviparum L.
Potygonum manshuriense V.Patr.
Polygonum alopecuroides Turcz.
Polygonum sphaerostachyum Meissn.
Andrographis paniculata (Burm.f.) Nees.
Taraxacum mongolicum Hand. and
Chrysanthemun indicumL.
Herb C is selected from the group consisting of:
Prunella vulgaris
Artemissia capillaris Thunb.
Gardenia jasminoides Ellis.
Rosa rugosa Thunb. and
Lophatherum gracile Brongn.;
Herb D is selected from the group consisting of:
Sonchus brachyotus
Patrinia scabiosaefolia Fisch.
Patrinia villosa Juss.
Sonchus arvensis L.
Thlaspi arvense L.
Portulaca oleracea L. and
Pulsatilla chinensis Reg. and
Herb E is selected from the group consisting of:
Dictamnus dasycarpus
Kochia scoparia (L.) Schard.
Sophora flavescens Ait. and
Heydyotis diffusa (Willd.) Roxbl
wherein, when the composition comprises three herbs, they are present in an amount from about 9% to about 57%; when the composition comprises four herbs, they are present in an amount from about 6% to about 43% or when the composition comprises four herbs, the first three are selected from the group consisting of Herb A, Herb B, Herb C and Herb D and are present in an amount from about 7% to about 57% and the fourth is selected fro m Herb E and is present in an amount from about 3.5% to about 28.5%; and when the composition comprises five herbs they are present in an amount from about 5% to about 35% or the first four herbs are selected from the group consisting of Herb A, Herb B, Herb C and Herb D and are present in an amount from about 6% to about 38% and the fifth herb is selected from Herb E and is present in an amount from about 3% to about 19%.

2. The composition of claim 1 wherein the composition comprises three herbs that are present in an amount from about 15% to 50%.

3. The composition of claim 2 wherein the three herbs are present in an amount from about 25% to about 40%.

4. The composition of claim 3 wherein each of the three herbs are present in an amount of about 33%.

5. The composition of claim 1 wherein the composition comprises four herbs where each are present in an amount of about 15% to about 35%.

6. The composition of claim 5 wherein the four herbs are present in an amount of about 25%.

7. The composition of claim 1 comprising four herbs wherein the first three are selected from the group consisting of Herb A, Herb B, Herb C and Herb D and are present in an amount from about 15% to about 48% and the fourth herb is selected from Herb E and is present in an amount from about 7.5% to about 24%.

8. The composition of claim 7 wherein the first three herbs are present in the composition in an amount of about 20% to about 43% and the fourth herb is present in an amount from about 10% to about 21.5%.

9. The composition of claim 8 wherein the first three herbs are present in an amount of about 28% and the fourth herb is present in an amount of about 14%.

10. The composition of claim 1 wherein the composition comprises five herbs and each are present in an amount from about 10% to about 30%.

11. The composition of claim 10 wherein the five herbs are present in an amount from about 15% to about 25%.

12. The composition of claim 11 wherein each of the five herbs are present in an amount of about 20%.

13. The composition of claim 1 wherein the composition comprises five herbs wherein the first four herbs are selected from the group consisting of Herb A, Herb B, Herb C and Herb D and is present in an amount from about 12% to about 32% and the fifth herb is selected from Herb E and is present in an amount from about 6% to about 16%.

14. The composition of claim 13 wherein the first four herbs are present in an amount from about 18% to about 26% and the fifth herb is present in an amount from about 9% to about 13%.

15. The composition of claim 14 wherein the first four herbs are present in an amount of about 22% whereas the fifth herb is present in an amount of about 11%.

16. The composition of claim 15 comprising a mixture of the following herbs: Sophora tonkinesis, Polygonum bistorta, Prunella vulgaris, Sonchus brachyotus and Dictamnus dasycarpus Turcz.

17. The composition of claim 9 comprising a mixture of the following herb: Sophora tonkinensis, Polygonum bistorta, Prunella vulgaris and Dictamnus dasycarpus.

18. The composition of claim 4 comprising a mixture of the following herbs: Sophora tonkinensis, Polygonum bistorta and Prunella vulgaris.

19. The composition of claim 1 further comprising a sixth herb, Herb F, selected from the group consisting of:
Dioscorea bulbifera
Panax notoginseng (Burk) F. H. Chen.
Bletilla striata (Thunb.) Reichb.f.
Nelumbo nucifera Gaertn.
Polygonum bistorta L.
Cephalanoplos segetum (Boge) Kitam.
Cirsium japonicum DC.
Sophora japonica L.
Typha angustfolia L. and
Rubia cordifolia L.;
wherein the first five herbs are selected from the group consisting of Herb A, Herb B, Herb C, Herb D and Herb E wherein the herbs are present in an amount from about 5% to about 31% and the sixth herb is selected from Herb F and is present in an amount from about 2.5% to about 17.5% or the first four herbs are selected from the group consisting of Herb A, Herb B, Herb C or Herb D and is present in an amount from about 5% to about 35% and the fifth is selected from Herb E and is present in an amount from about 2.5% to about 19% and the sixth herb is selected from Herb F and is present in an amount from about 1.75% to about 9.5%.

20. The composition of claim 19 wherein the first five herbs are selected from Herb A, Herb B, Herb C, Herb D and Herb E and are present in an amount from about 10% to about 26% and the sixth herb is selected from Herb F and is present in an amount from about 5% to about 15%.

21. The composition of claim 20 wherein the first five herbs are present in an amount from about 15% to about 21% and the sixth herb is present in an amount from about 7.5% to about 12.5%.

22. The composition of claim 21 wherein the first five herbs are present in an amount of about 18% and the sixth herb is present in an amount of about 10%.

23. The composition of claim 19 wherein the first four herbs are selected from the group consisting of Herb A, Herb B, Herb C or Herb D and are present in an amount from about 10% to about 33% and the fifth herb is selected from Herb E and is present in an amount of about 5% to about 16.5% and the sixth herb is selected from Herb F and is present in an amount of about 3% to about 8.25%.

24. The composition of claim 23 wherein the first four herbs are present in an amount of about 15% to about 28% and Herb five is present in an amount of about 7.5% to about 14.5% and the sixth herb is present in an amount of about 4.25% to about 7%.

25. The composition of claim 24 wherein the first four herbs are present in an amount of about 21% and the fifth herb is present in an amount of about 10.5% and the sixth herb is present in an amount of about 5.5%.

26. The composition of claim 24 comprising a mixture of the following herbs: Sophora tonkinensis, Polygonum bistorta, Prunella vulgaris, Sonchus brachyotus, Dictamnus dasycarpus Turcz, and Dioscorea bulbifera.

27. A method of preparing the composition as defined in claim 1 comprising the steps of:
subjected the herbs to an aqueous extraction,
optionally filtering the extract to remove large particles,
drying the aqueous extract; and formulating the dried extract into a form suitable for administration.

28. The method of claim 27 wherein the method further comprises using one or more of the herbs directly in powdered form, without preparing an aqueous extract.

29. A method of preventing or treating cancer by administering to a patient in need thereof an effective amount of a composition as defined in claim 1.

30. The method of claim 29 wherein the cancer is selected from the group selected from cancer of the lung, esophagus, stomach, oral cavity and prostate.

31. A method of treating Helicobacter pylori infection by administering to a patient in need thereof an effective amount of a composition as defined in claim 1.

32. A method of treating and reducing the reoccurrence of cancer by administering to a patient in need thereof an effective amount of a composition as defined in claim 1, as adjuncts to conventional surgery or radiotherapy treatments.

33. The method of any of claims 29 to 32, wherein the composition is administered at a daily dose of from about 1.2 grams to about 6.0 grams.

34. A method of preparing the composition as defined in claim 19 comprising the steps of:
subjected the herbs to an aqueous extraction,
optionally filtering the extract to remove large particles,
drying the aqueous extract; and formulating the dried extract into a form suitable for administration.

35. The method of claim 34 wherein the method further comprises using one or more of the herbs directly in powdered form, without preparing an aqueous extract.

36. A method of preventing or treating cancer by administering to a patient in need thereof an effective amount of a composition as defined in claim 19.

37. The method of claim 38 wherein the cancer is selected from the group selected from cancer of the lung, esophagus, stomach, oral cavity and prostate.

38. A method of treating Helicobacter pylori infection by administering to a patient in need thereof an effective amount of a composition as defined in claim 19.

39. A method of treating and reducing the reoccurrence of cancer by administering to a patient in need thereof an effective amount of a composition as defined in claim 19, as adjuncts to conventional surgery or radiotherapy treatments.

40. The method of any of claims 36 to 39, wherein the composition is administered at a daily dose of from about 1.2 grams to about 6.0 grams.
